SEQUENCE LISTING (146 sequences)

SEQ ID NO: 1 - DNA, 2196 bp, Equus caballus
  source 1..2196: mol_type = other DNA
  CDS 39..1463: protein_id = 143; transl_table = 1; translation = MNGYGSPYLYMGGPVSQPPRAPLQRTPKCARCRNHGVLSWLKGH KRYCRFKDCTCEKCILIIERQRVMAAQVALRRQQANESLESLIPDSLRALPGPPPPGD AAAAAPQPPPTSQPSQPPPPQRPAAELAAAAALRWATEPQPGALQAQLDKPDLTEERL GDGSSADNTETFSDKDTDQRSSPDVVKSKGCFTPESPEVVSVDEGGYAVQKNGGTSES RPDSPKYHGEQNHLLIEGPSGTVSLPFSLKANRPPLEVLKKIFPNQKPTVLELILKGC GGDLVSAVEVLLSSRSSASAADRTSAEPESLVLPSNGHIFEHTLSSYPISSSKWSVGS AFRVPDTLRFSADSSNVVPNPLAVPLQHPFPQPPRYPLMLRNTLARNQSSPFLPNDVT LWNTMTLQQQYQLRSQYVSPFPGSSPSVFRSSPVLPTRAPEDPRISIPDDGCPIVSKQ SLYTEDDYDERSDSSDSRILNTSS
ccgccgccagcccgccagctcttccgggagctcagggcatgaacggctacggttccccctacctgtacatgggcggcccggtgtcgcagccgccgcgggcgcccttgcagcgcacgcccaagtgcgcgcgctgccgcaaccacggggtgctgtcctggctcaagggtcacaagcgctactgccgcttcaaggactgcacctgcgagaagtgcatcctcatcatcgagcggcagagggtcatggcggcgcaggtggcgctgcgccggcagcaagctaacgagagcctcgagagcctcattcccgactcgctgcgtgctctgcccggccccccgccgccgggggacgccgccgctgccgccccgcagccgccgcccacctcgcagccgtctcagccgccgccgccgcagcgtcccgccgccgagttggctgcggccgccgcgctgcgctgggccaccgagccgcagcccggggcgctgcaggcgcagctcgacaagccagatttgactgaggagcgacttggggacggcagctccgcagacaacacagagaccttcagcgacaaagacaccgaccagaggagctccccagatgtggtgaaaagtaagggctgcttcaccccggagagccccgaggtcgtgtctgtggatgaaggcgggtatgcggtccagaagaacggaggcacctccgagagccgccccgacagtcccaagtaccacggggaacagaatcacctcctgatcgagggcccctcggggaccgtttctctgcccttcagcttgaaagccaacagaccgcccctggaagtgttaaaaaaaatcttccccaaccagaagcccacggtgctggagctcatcctgaagggctgtgggggcgacctggtgagcgccgtggaggtcctcctctccagccgctcctcggcctcggccgccgaccgaacttcggcagagcccgagagcctcgtgttgccctccaacgggcacatctttgaacacaccttgagctcataccccatctcctcttccaaatggtccgtgggatcggccttcagggtcccagacacgttgaggttttccgcagactctagtaacgttgtccccaaccccttggccgtgcccctgcagcatcctttcccccagccgccccggtaccctctgatgctgaggaatactttggcaagaaaccagtcgagccccttcctgcccaatgatgtcaccctgtggaacaccatgacgctgcagcagcagtaccagctgaggtcccagtacgtcagccctttccccgggagctcgcccagcgtcttcagaagctcgcctgtccttcccacgcgcgcccccgaagaccctcggatctccatccctgacgatgggtgtccgattgtgtcaaagcagtctctttacaccgaggatgactatgacgagaggtccgactcctcagactctagaatactcaacacatcatcttaaagtggtaccgggtggctggtgaccaggtgacattttctgtgcatttgaactctgaccccctgccctccccaggagaggcctcgtcctgtgtataccctttccttctgtttgacaaagtgactgtgcttgattctataccttagcaataaaaacataacttatttaatttcttgcacttcactggaaaatgccaaatagctctgctctgcggctttagtgctgaatgtttattgtaaaagagagtctaatgctaagaatagtcttgggaaagctgggtccacggaagatttatttggggatggaaagctgaaggtcagccttgctcctaaactcaacctggaatgttcaataaaatagtatacttgaatgcagttttgtaaaaaaggattcctcaggatatttgaaacctaaaggaagtggtttggttgcaaatggaccagaaacagggacattatattcttaggctaaaaaccttgcatttaaaagagagactgcacttaagaatagagtgaactgctcacatgcttatttaagcttggacagttttcagagacaaattccattaagaattattcttttcacatgaccgaatcgaaacatgtgtaatgtcaatgtaaaaccaatcacagctgtgaactgcatgaaatgtattgtgaaatgaacacaagattaagctttgtcaggttaatgtagcatgctaaggactctagaaaaaaataaactaaggagatga

SEQ ID NO: 2 - protein, 474 aa, Equus caballus
MNGYGSPYLYMGGPVSQPPRAPLQRTPKCARCRNHGVLSWLKGHKRYCRFKDCTCEKCILIIERQRVMAAQVALRRQQANESLESLIPDSLRALPGPPPPGDAAAAAPQPPPTSQPSQPPPPQRPAAELAAAAALRWATEPQPGALQAQLDKPDLTEERLGDGSSADNTETFSDKDTDQRSSPDVVKSKGCFTPESPEVVSVDEGGYAVQKNGGTSESRPDSPKYHGEQNHLLIEGPSGTVSLPFSLKANRPPLEVLKKIFPNQKPTVLELILKGCGGDLVSAVEVLLSSRSSASAADRTSAEPESLVLPSNGHIFEHTLSSYPISSSKWSVGSAFRVPDTLRFSADSSNVVPNPLAVPLQHPFPQPPRYPLMLRNTLARNQSSPFLPNDVTLWNTMTLQQQYQLRSQYVSPFPGSSPSVFRSSPVLPTRAPEDPRISIPDDGCPIVSKQSLYTEDDYDERSDSSDSRILNTSS

SEQ ID NO: 3 - DNA, 2196 bp, Equus caballus
  source 1..2196: mol_type = other DNA
  CDS 39..941: protein_id = 144; transl_table = 1; translation = MNGYGSPYLYMGGPVSQPPRAPLQRTPKCARCRNHGVLSWLKGH KRYCRFKDCTCEKCILIIERQRVMAAQVALRRQQANESLESLIPDSLRALPGPPPPGD AAAAAPQPPPTSQPSQPPPPQRPAAELAAAAALRWATEPQPGALQAQLDKPDLTEERL GDGSSADNTETFSDKDTDQRSSPDVVKSKGCFTPESPEVVSVDEGGYAVQKNGGTSES RPDSPKYHGEQNHLLIEGPSGTVSLPFSLKANRPPLEVLKKIFPNQKPTVLELILKGC GGDLVSAVEVLLSSRSSASAADRT
ccgccgccagcccgccagctcttccgggagctcagggcatgaacggctacggttccccctacctgtacatgggcggcccggtgtcgcagccgccgcgggcgcccttgcagcgcacgcccaagtgcgcgcgctgccgcaaccacggggtgctgtcctggctcaagggtcacaagcgctactgccgcttcaaggactgcacctgcgagaagtgcatcctcatcatcgagcggcagagggtcatggcggcgcaggtggcgctgcgccggcagcaagctaacgagagcctcgagagcctcattcccgactcgctgcgtgctctgcccggccccccgccgccgggggacgccgccgctgccgccccgcagccgccgcccacctcgcagccgtctcagccgccgccgccgcagcgtcccgccgccgagttggctgcggccgccgcgctgcgctgggccaccgagccgcagcccggggcgctgcaggcgcagctcgacaagccagatttgactgaggagcgacttggggacggcagctccgcagacaacacagagaccttcagcgacaaagacaccgaccagaggagctccccagatgtggtgaaaagtaagggctgcttcaccccggagagccccgaggtcgtgtctgtggatgaaggcgggtatgcggtccagaagaacggaggcacctccgagagccgccccgacagtcccaagtaccacggggaacagaatcacctcctgatcgagggcccctcggggaccgtttctctgcccttcagcttgaaagccaacagaccgcccctggaagtgttaaaaaaaatcttccccaaccagaagcccacggtgctggagctcatcctgaagggctgtgggggcgacctggtgagcgccgtggaggtcctcctctccagccgctcctcggcctcggccgccgaccgaacttaggcagagcccgagagcctcgtgttgccctccaacgggcacatctttgaacacaccttgagctcataccccatctcctcttccaaatggtccgtgggatcggccttcagggtcccagacacgttgaggttttccgcagactctagtaacgttgtccccaaccccttggccgtgcccctgcagcatcctttcccccagccgccccggtaccctctgatgctgaggaatactttggcaagaaaccagtcgagccccttcctgcccaatgatgtcaccctgtggaacaccatgacgctgcagcagcagtaccagctgaggtcccagtacgtcagccctttccccgggagctcgcccagcgtcttcagaagctcgcctgtccttcccacgcgcgcccccgaagaccctcggatctccatccctgacgatgggtgtccgattgtgtcaaagcagtctctttacaccgaggatgactatgacgagaggtccgactcctcagactctagaatactcaacacatcatcttaaagtggtaccgggtggctggtgaccaggtgacattttctgtgcatttgaactctgaccccctgccctccccaggagaggcctcgtcctgtgtataccctttccttctgtttgacaaagtgactgtgcttgattctataccttagcaataaaaacataacttatttaatttcttgcacttcactggaaaatgccaaatagctctgctctgcggctttagtgctgaatgtttattgtaaaagagagtctaatgctaagaatagtcttgggaaagctgggtccacggaagatttatttggggatggaaagctgaaggtcagccttgctcctaaactcaacctggaatgttcaataaaatagtatacttgaatgcagttttgtaaaaaaggattcctcaggatatttgaaacctaaaggaagtggtttggttgcaaatggaccagaaacagggacattatattcttaggctaaaaaccttgcatttaaaagagagactgcacttaagaatagagtgaactgctcacatgcttatttaagcttggacagttttcagagacaaattccattaagaattattcttttcacatgaccgaatcgaaacatgtgtaatgtcaatgtaaaaccaatcacagctgtgaactgcatgaaatgtattgtgaaatgaacacaagattaagctttgtcaggttaatgtagcatgctaaggactctagaaaaaaataaactaaggagatga

SEQ ID NO: 4 - protein, 300 aa, Equus caballus
MNGYGSPYLYMGGPVSQPPRAPLQRTPKCARCRNHGVLSWLKGHKRYCRFKDCTCEKCILIIERQRVMAAQVALRRQQANESLESLIPDSLRALPGPPPPGDAAAAAPQPPPTSQPSQPPPPQRPAAELAAAAALRWATEPQPGALQAQLDKPDLTEERLGDGSSADNTETFSDKDTDQRSSPDVVKSKGCFTPESPEVVSVDEGGYAVQKNGGTSESRPDSPKYHGEQNHLLIEGPSGTVSLPFSLKANRPPLEVLKKIFPNQKPTVLELILKGCGGDLVSAVEVLLSSRSSASAADRT

SEQ ID NO: 5 - DNA, 101 bp, Equus caballus
  source 1..101: mol_type = other DNA
ttgttggggtcttatgcaaagtgcagaaatagtcttttggaaaaacgtacygtttgcactattttcttatttctattcacccttaatgtgaaccattccaa

SEQ ID NO: 6 - DNA, 101 bp, Equus caballus
  source 1..101: mol_type = other DNA
agaaatgatatataaaaattacgaatgcctcttagacagaatccttatgtrtggcacagaagtatttagttcgcttaacagatattgagtgcttatatgag

SEQ ID NO: 7 - DNA, 101 bp, Equus caballus
  source 1..101: mol_type = other DNA
ctcttccttgcatcctatccccctagtgtcgcaagggaagttgtgagagaygagcttgtagatctgctctagaaaataggcctgttttcttaagaaaccgt

SEQ ID NO: 8 - DNA, 101 bp, Equus caballus
  source 1..101: mol_type = other DNA
cagagtgccggtctgtggctgtgggcgctgacgagcacatggacaacattstcgtgtaccacaggggctccaggtcctgtaaggatgctgctgtggggaca

SEQ ID NO: 9 - DNA, 101 bp, Equus caballus
  source 1..101: mol_type = other DNA
agaactcattcaaaaccaccaggcttactaggcttttttaaatagacttgrctttgaacttctaagtgcaggatctaaaaccactggcgaaatttctggaa

SEQ ID NO: 10 - DNA, 101 bp, Equus caballus
  source 1..101: mol_type = other DNA
ttacctgcatgcctctccccctaaaccatttctagcatgtgtgggcagagkgggcatcgtgctgccctgctcactggatcactctgggaacgtttccttca

SEQ ID NO: 11 - DNA, 101 bp, Equus caballus
  source 1..101: mol_type = other DNA
aaggatatggtgagtctgacctacagacactgtccccggtctgtacaaagygcccaagtggtgacaaagcatccctcgcctgccccctgagctgttacctg

SEQ ID NO: 12 - DNA, 101 bp, Equus caballus
  source 1..101: mol_type = other DNA
aacgccaaagccagccaggtgactgcgcttgcttcctgggctcatgctcayactgctgtgacccgcacaggtgcccacgccacacttcccaccgctcggca

SEQ ID NO: 13 - DNA, 101 bp, Equus caballus
  source 1..101: mol_type = other DNA
gcttgcttcctgggctcatgctcacactgctgtgacccgcacaggtgcccrcgccacacttcccaccgctcggcactcactcatggcccagccccgagtcc

SEQ ID NO: 14 - DNA, 101 bp, Equus caballus
  source 1..101: mol_type = other DNA
actgaatgtatacattttgtgcctgaactcaccagcaaacagaaggcagamaaccaagggttgaaggctggagctgtcacagtagaagttgagccagcagg

SEQ ID NO: 15 - DNA, 101 bp, Equus caballus
  source 1..101: mol_type = other DNA
cattttgtgcctgaactcaccagcaaacagaaggcagaaaaccaagggttsaaggctggagctgtcacagtagaagttgagccagcaggaatttgctggcc

SEQ ID NO: 16 - DNA, 101 bp, Equus caballus
  source 1..101: mol_type = other DNA
tcactctaatcaagttgctatcaccattcacacaattgtccaggatagtaytgggaccccagaaagatcacgccgctccattcccatttcccacttgttcc

SEQ ID NO: 17 - DNA, 101 bp, Equus caballus
  source 1..101: mol_type = other DNA
ctgggctgaaacaggtggtcctgctttccccgcctgcctggtcaggctgcrctcttctcccctccccaggcttaagtcacttcatgcagaaccctttatac

SEQ ID NO: 18 - DNA, 101 bp, Equus caballus
  source 1..101: mol_type = other DNA
ccagcattctccgctttcaactttctcccgctcctccaatccaaactggasttagcatcagctacccacaatgatcaagcattttctgtgtggcaggcctg

SEQ ID NO: 19 - DNA, 101 bp, Equus caballus
  source 1..101: mol_type = other DNA
aggcaagaagcgataggcaaaaggcagcaagagctggacctgcagatttgmaagttctctggagccagtaggtggaaacctcatcagcaaatgaacgcagg

SEQ ID NO: 20 - DNA, 101 bp, Equus caballus
  source 1..101: mol_type = other DNA
ccacactgagagtcttatttgctgatagaaatgcagagacttctcttttcygaggctttcaacctcgtacttaattctcctaagtgagaaagaaaccactc

SEQ ID NO: 21 - DNA, 101 bp, Equus caballus
  source 1..101: mol_type = other DNA
accagcgggagactgaggctgcgagcgccgcaaagacgggtgccgcatctytggccagcccggagcgcacgcggccgccggagctgcgggaccaaggaccg

SEQ ID NO: 22 - DNA, 101 bp, Equus caballus
  source 1..101: mol_type = other DNA
ccgtctcagccgccgccgccgcagcgtcccgccgccgagttggctgcggcygccgcgctgcgctgggccaccgagccgcagcccggggcgctgcaggcgca

SEQ ID NO: 23 - DNA, 101 bp, Equus caballus
  source 1..101: mol_type = other DNA
ggaggtcctcctctccagccgctcctcggcctcggccgccgaccgaacttmggcagagcccgagagcctcgtgttgccctccaacgggcacatctttgaac

SEQ ID NO: 24 - DNA, 101 bp, Equus caballus
  source 1..101: mol_type = other DNA
ctctccagccgctcctcggcctcggccgccgaccgaacttcggcagagccsgagagcctcgtgttgccctccaacgggcacatctttgaacacaccttgag

SEQ ID NO: 25 - DNA, 101 bp, Equus caballus
  source 1..101: mol_type = other DNA
ggcctggcccctagggcattgaagggctggggagagtcacatgtactcccwctgtggcctgaagacctacctggagggaaaccagcttgcttagggggcct

SEQ ID NO: 26 - DNA, 26 bp, synthetic construct
  misc_feature 1..26: note = Primer
  source 1..26: mol_type = other DNA
gcaaagtgcagaaatagtcttttgga

SEQ ID NO: 27 - DNA, 28 bp, synthetic construct
  misc_feature 1..28: note = Primer
  source 1..28: mol_type = other DNA
cactcttttggaatggttcacattaagg

SEQ ID NO: 28 - DNA, 18 bp, synthetic construct
  misc_feature 1..18: note = Reporter
  source 1..18: mol_type = other DNA
tagtgcaaacggtacgtt

SEQ ID NO: 29 - DNA, 21 bp, synthetic construct
  misc_feature 1..21: note = Reporter
  source 1..21: mol_type = other DNA
aaatagtgcaaacagtacgtt

SEQ ID NO: 30 - DNA, 17 bp, synthetic construct
  misc_feature 1..17: note = Primer
  source 1..17: mol_type = other DNA
cctctccagccgctcct

SEQ ID NO: 31 - DNA, 20 bp, synthetic construct
  misc_feature 1..20: note = Primer
  source 1..20: mol_type = other DNA
tcaaagatgtgcccgttgga

SEQ ID NO: 32 - DNA, 13 bp, synthetic construct
  misc_feature 1..13: note = Reporter
  source 1..13: mol_type = other DNA
ctgccgaagttcg

SEQ ID NO: 33 - DNA, 15 bp, synthetic construct
  misc_feature 1..15: note = Reporter
  source 1..15: mol_type = other DNA
ctctgcctaagttcg

SEQ ID NO: 34 - DNA, 32 bp, synthetic construct
  misc_feature 1..32: note = Probe
  source 1..32: mol_type = other DNA
ggatcctcgagaacaatgtaacaatttcgccc

SEQ ID NO: 35 - DNA, 19 bp, synthetic construct
  misc_feature 1..19: note = Primer
  source 1..19: mol_type = other DNA
cataccagcttgccacact

SEQ ID NO: 36 - DNA, 20 bp, synthetic construct
  misc_feature 1..20: note = Primer
  source 1..20: mol_type = other DNA
gaggagagagagctcgtgga

SEQ ID NO: 37 - DNA, 20 bp, synthetic construct
  misc_feature 1..20: note = Primer
  source 1..20: mol_type = other DNA
ctaatggagacccgcagaag

SEQ ID NO: 38 - DNA, 20 bp, synthetic construct
  misc_feature 1..20: note = Primer
  source 1..20: mol_type = other DNA
gccggaactcctttatcctc

SEQ ID NO: 39 - DNA, 20 bp, synthetic construct
  misc_feature 1..20: note = Primer
  source 1..20: mol_type = other DNA
gagaagtggcggggaattat

SEQ ID NO: 40 - DNA, 20 bp, synthetic construct
  misc_feature 1..20: note = Primer
  source 1..20: mol_type = other DNA
gccccacgactttattctca

SEQ ID NO: 41 - DNA, 20 bp, synthetic construct
  misc_feature 1..20: note = Primer
  source 1..20: mol_type = other DNA
tgcagacgagagaccaaatg

SEQ ID NO: 42 - DNA, 20 bp, synthetic construct
  misc_feature 1..20: note = Primer
  source 1..20: mol_type = other DNA
aaacccagaagtgcctgaga

SEQ ID NO: 43 - DNA, 20 bp, synthetic construct
  misc_feature 1..20: note = Primer
  source 1..20: mol_type = other DNA
gcggacagtggctataggag

SEQ ID NO: 44 - DNA, 21 bp, synthetic construct
  misc_feature 1..21: note = Primer
  source 1..21: mol_type = other DNA
aatacattgtccccacccttc

SEQ ID NO: 45 - DNA, 20 bp, synthetic construct
  misc_feature 1..20: note = Primer
  source 1..20: mol_type = other DNA
atgggatttgagctgagtgg

SEQ ID NO: 46 - DNA, 20 bp, synthetic construct
  misc_feature 1..20: note = Primer
  source 1..20: mol_type = other DNA
aagcctgatgctgagaagga

SEQ ID NO: 47 - DNA, 20 bp, synthetic construct
  misc_feature 1..20: note = Primer
  source 1..20: mol_type = other DNA
ttgcatgcacacaattttcc

SEQ ID NO: 48 - DNA, 20 bp, synthetic construct
  misc_feature 1..20: note = Primer
  source 1..20: mol_type = other DNA
ctgggggtttctgagttctg

SEQ ID NO: 49 - DNA, 20 bp, synthetic construct
  misc_feature 1..20: note = Primer
  source 1..20: mol_type = other DNA
gcaacccaggttatcccttt

SEQ ID NO: 50 - DNA, 20 bp, synthetic construct
  misc_feature 1..20: note = Primer
  source 1..20: mol_type = other DNA
tcaccttctgcacttgcatt

SEQ ID NO: 51 - DNA, 20 bp, synthetic construct
  misc_feature 1..20: note = Primer
  source 1..20: mol_type = other DNA
aagtcgactgaggggctctt

SEQ ID NO: 52 - DNA, 20 bp, synthetic construct
  misc_feature 1..20: note = Primer
  source 1..20: mol_type = other DNA
accttggcccagataggttt

SEQ ID NO: 53 - DNA, 20 bp, synthetic construct
  misc_feature 1..20: note = Primer
  source 1..20: mol_type = other DNA
tccccaggaacatacagctc

SEQ ID NO: 54 - DNA, 20 bp, synthetic construct
  misc_feature 1..20: note = Primer
  source 1..20: mol_type = other DNA
tggaaaggatttgaggatgc

SEQ ID NO: 55 - DNA, 20 bp, synthetic construct
  misc_feature 1..20: note = Primer
  source 1..20: mol_type = other DNA
gcttctggcctcacgaaata

SEQ ID NO: 56 - DNA, 20 bp, synthetic construct
  misc_feature 1..20: note = Primer
  source 1..20: mol_type = other DNA
tggcatgaagacaccacaat

SEQ ID NO: 57 - DNA, 20 bp, synthetic construct
  misc_feature 1..20: note = Primer
  source 1..20: mol_type = other DNA
agccccagtacagaccacac

SEQ ID NO: 58 - DNA, 20 bp, synthetic construct
  misc_feature 1..20: note = Primer
  source 1..20: mol_type = other DNA
gggaagtcgcctacactgaa

SEQ ID NO: 59 - DNA, 20 bp, synthetic construct
  misc_feature 1..20: note = Primer
  source 1..20: mol_type = other DNA
gaggatccgtgggatacaga

SEQ ID NO: 60 - DNA, 20 bp, synthetic construct
  misc_feature 1..20: note = Primer
  source 1..20: mol_type = other DNA
agcaagtctcctgagcaagc

SEQ ID NO: 61 - DNA, 20 bp, synthetic construct
  misc_feature 1..20: note = Primer
  source 1..20: mol_type = other DNA
cagaggacacatctgcctga

SEQ ID NO: 62 - DNA, 20 bp, synthetic construct
  misc_feature 1..20: note = Primer
  source 1..20: mol_type = other DNA
caaaaccatcctggaaatgg

SEQ ID NO: 63 - DNA, 20 bp, synthetic construct
  misc_feature 1..20: note = Primer
  source 1..20: mol_type = other DNA
agactggccctgagctaaca

SEQ ID NO: 64 - DNA, 20 bp, synthetic construct
  misc_feature 1..20: note = Primer
  source 1..20: mol_type = other DNA
tgtcagctgcagaatggaag

SEQ ID NO: 65 - DNA, 20 bp, synthetic construct
  misc_feature 1..20: note = Primer
  source 1..20: mol_type = other DNA
agactggccctgagctaaca

SEQ ID NO: 66 - DNA, 20 bp, synthetic construct
  misc_feature 1..20: note = Primer
  source 1..20: mol_type = other DNA
ctgaaggtgccctctacagc

SEQ ID NO: 67 - DNA, 20 bp, synthetic construct
  misc_feature 1..20: note = Primer
  source 1..20: mol_type = other DNA
ttacctgcccctttgttttg

SEQ ID NO: 68 - DNA, 20 bp, synthetic construct
  misc_feature 1..20: note = Primer
  source 1..20: mol_type = other DNA
catctttgcccctcagactc

SEQ ID NO: 69 - DNA, 20 bp, synthetic construct
  misc_feature 1..20: note = Primer
  source 1..20: mol_type = other DNA
ttacgtggcacccctacttc

SEQ ID NO: 70 - DNA, 20 bp, synthetic construct
  misc_feature 1..20: note = Primer
  source 1..20: mol_type = other DNA
agcctggactctgtccttga

SEQ ID NO: 71 - DNA, 20 bp, synthetic construct
  misc_feature 1..20: note = Primer
  source 1..20: mol_type = other DNA
tgctgccctctgtctatgtg

SEQ ID NO: 72 - DNA, 20 bp, synthetic construct
  misc_feature 1..20: note = Primer
  source 1..20: mol_type = other DNA
aaagtaacgatgcggtggac

SEQ ID NO: 73 - DNA, 20 bp, synthetic construct
  misc_feature 1..20: note = Primer
  source 1..20: mol_type = other DNA
aaatggctgtgccgttttac

SEQ ID NO: 74 - DNA, 20 bp, synthetic construct
  misc_feature 1..20: note = Primer
  source 1..20: mol_type = other DNA
ctgtgtgaccaagctctcca

SEQ ID NO: 75 - DNA, 20 bp, synthetic construct
  misc_feature 1..20: note = Primer
  source 1..20: mol_type = other DNA
gaaaatgctgacgtgctgaa

SEQ ID NO: 76 - DNA, 20 bp, synthetic construct
  misc_feature 1..20: note = Primer
  source 1..20: mol_type = other DNA
cttgctgccttttgcctatc

SEQ ID NO: 77 - DNA, 20 bp, synthetic construct
  misc_feature 1..20: note = Primer
  source 1..20: mol_type = other DNA
gcagagcgacctggagatag

SEQ ID NO: 78 - DNA, 20 bp, synthetic construct
  misc_feature 1..20: note = Primer
  source 1..20: mol_type = other DNA
ggccttagagggacacatga

SEQ ID NO: 79 - DNA, 20 bp, synthetic construct
  misc_feature 1..20: note = Primer
  source 1..20: mol_type = other DNA
cctctcacccagacaccatt

SEQ ID NO: 80 - DNA, 20 bp, synthetic construct
  misc_feature 1..20: note = Primer
  source 1..20: mol_type = other DNA
agttggcaaacaacaggaca

SEQ ID NO: 81 - DNA, 20 bp, synthetic construct
  misc_feature 1..20: note = Primer
  source 1..20: mol_type = other DNA
aagtcctttcttgggggcta

SEQ ID NO: 82 - DNA, 20 bp, synthetic construct
  misc_feature 1..20: note = Primer
  source 1..20: mol_type = other DNA
ggtccatcgttgaccaaaat

SEQ ID NO: 83 - DNA, 20 bp, synthetic construct
  misc_feature 1..20: note = Primeer
  source 1..20: mol_type = other DNA
agtcctttcttgggggctaa

SEQ ID NO: 84 - DNA, 20 bp, synthetic construct
  misc_feature 1..20: note = Primer
  source 1..20: mol_type = other DNA
acggcaccaccatcatctat

SEQ ID NO: 85 - DNA, 20 bp, synthetic construct
  misc_feature 1..20: note = Primer
  source 1..20: mol_type = other DNA
gccccaacttaagaccctct

SEQ ID NO: 86 - DNA, 18 bp, synthetic construct
  misc_feature 1..18: note = Primer
  source 1..18: mol_type = other DNA
ccgcgctgcttaggagtc

SEQ ID NO: 87 - DNA, 20 bp, synthetic construct
  misc_feature 1..20: note = Primer
  source 1..20: mol_type = other DNA
gccccaacttaagaccctct

SEQ ID NO: 88 - DNA, 19 bp, synthetic construct
  misc_feature 1..19: note = Primer
  source 1..19: mol_type = other DNA
tacctggcttgtcgagctg

SEQ ID NO: 89 - DNA, 20 bp, synthetic construct
  misc_feature 1..20: note = Primer
  source 1..20: mol_type = other DNA
gagcacgctcagaccctatc

SEQ ID NO: 90 - DNA, 21 bp, synthetic construct
  misc_feature 1..21: note = Primer
  source 1..21: mol_type = other DNA
aaagagctccgaagtttttgc

SEQ ID NO: 91 - DNA, 20 bp, synthetic construct
  misc_feature 1..20: note = Primer
  source 1..20: mol_type = other DNA
ctccttccaagaagcctgtg

SEQ ID NO: 92 - DNA, 20 bp, synthetic construct
  misc_feature 1..20: note = Primer
  source 1..20: mol_type = other DNA
agagtctgcggaaaacctca

SEQ ID NO: 93 - DNA, 20 bp, synthetic construct
  misc_feature 1..20: note = Primer
  source 1..20: mol_type = other DNA
ccttgagctcataccccatc

SEQ ID NO: 94 - DNA, 20 bp, synthetic construct
  misc_feature 1..20: note = Primer
  source 1..20: mol_type = other DNA
actaaagccgcagagcagag

SEQ ID NO: 95 - DNA, 20 bp, synthetic construct
  misc_feature 1..20: note = Primer
  source 1..20: mol_type = other DNA
gagaggcctcgtcctgtgta

SEQ ID NO: 96 - DNA, 20 bp, synthetic construct
  misc_feature 1..20: note = Primer
  source 1..20: mol_type = other DNA
tcccactcacatttcccaat

SEQ ID NO: 97 - DNA, 20 bp, synthetic construct
  misc_feature 1..20: note = Primer
  source 1..20: mol_type = other DNA
caagggcatgaggagtgttt

SEQ ID NO: 98 - DNA, 20 bp, synthetic construct
  misc_feature 1..20: note = Primer
  source 1..20: mol_type = other DNA
actccatgattgcacaacga

SEQ ID NO: 99 - DNA, 20 bp, synthetic construct
  misc_feature 1..20: note = Primer
  source 1..20: mol_type = other DNA
tcattccaccagcaatgtgt

SEQ ID NO: 100 - DNA, 20 bp, synthetic construct
  misc_feature 1..20: note = Primer
  source 1..20: mol_type = other DNA
ggccactgcagaagaaagag

SEQ ID NO: 101 - DNA, 20 bp, synthetic construct
  misc_feature 1..20: note = Primer
  source 1..20: mol_type = other DNA
ctgttgtcccagccctgtat

SEQ ID NO: 102 - DNA, 20 bp, synthetic construct
  misc_feature 1..20: note = Primer
  source 1..20: mol_type = other DNA
aggtgagtccaggctagcaa

SEQ ID NO: 103 - DNA, 20 bp, synthetic construct
  misc_feature 1..20: note = Primer
  source 1..20: mol_type = other DNA
gagcccgagcggataatact

SEQ ID NO: 104 - DNA, 20 bp, synthetic construct
  misc_feature 1..20: note = Primer
  source 1..20: mol_type = other DNA
attaggaccgcacaggacac

SEQ ID NO: 105 - DNA, 20 bp, synthetic construct
  misc_feature 1..20: note = Primer
  source 1..20: mol_type = other DNA
gcggctagggtggtacttct

SEQ ID NO: 106 - DNA, 18 bp, synthetic construct
  misc_feature 1..18: note = Primeer
  source 1..18: mol_type = other DNA
ctcgtcctcgtcctcgtc

SEQ ID NO: 107 - DNA, 18 bp, synthetic construct
  misc_feature 1..18: note = Primer
  source 1..18: mol_type = other DNA
gaggacgacgaggacgag

SEQ ID NO: 108 - DNA, 20 bp, synthetic construct
  misc_feature 1..20: note = primer
  source 1..20: mol_type = other DNA
ccactttcaaggcctctctg

SEQ ID NO: 109 - DNA, 18 bp, synthetic construct
  misc_feature 1..18: note = Primer
  source 1..18: mol_type = other DNA
gaggacgacgaggacgag

SEQ ID NO: 110 - DNA, 20 bp, synthetic construct
  misc_feature 1..20: note = Primer
  source 1..20: mol_type = other DNA
ccactttcaaggcctctctg

SEQ ID NO: 111 - DNA, 20 bp, synthetic construct
  misc_feature 1..20: note = Primer
  source 1..20: mol_type = other DNA
ctggggtgactctagcaagg

SEQ ID NO: 112 - DNA, 20 bp, synthetic construct
  misc_feature 1..20: note = Primer
  source 1..20: mol_type = other DNA
tcacaccaaggcaaatttca

SEQ ID NO: 113 - DNA, 20 bp, synthetic construct
  misc_feature 1..20: note = Primer
  source 1..20: mol_type = other DNA
cccccaaagggaactatttt

SEQ ID NO: 114 - DNA, 20 bp, synthetic construct
  misc_feature 1..20: note = Primer
  source 1..20: mol_type = other DNA
gaactgaggtggtggcattt

SEQ ID NO: 115 - DNA, 20 bp, synthetic construct
  misc_feature 1..20: note = Primer
  source 1..20: mol_type = other DNA
ttcagggtctgggaatatgg

SEQ ID NO: 116 - DNA, 20 bp, synthetic construct
  misc_feature 1..20: note = Primer
  source 1..20: mol_type = other DNA
tccaacttgtttggctacga

SEQ ID NO: 117 - DNA, 20 bp, synthetic construct
  misc_feature 1..20: note = Primer
  source 1..20: mol_type = other DNA
ggcccctaagaaacacagag

SEQ ID NO: 118 - DNA, 20 bp, synthetic construct
  misc_feature 1..20: note = Primer
  source 1..20: mol_type = other DNA
cctgtagaccccagagacca

SEQ ID NO: 119 - DNA, 20 bp, synthetic construct
  misc_feature 1..20: note = Primer
  source 1..20: mol_type = other DNA
ggtccaaattgtagggctga

SEQ ID NO: 120 - DNA, 20 bp, synthetic construct
  misc_feature 1..20: note = Primeer
  source 1..20: mol_type = other DNA
ttccccaggaggttctcttt

SEQ ID NO: 121 - DNA, 20 bp, synthetic construct
  misc_feature 1..20: note = Primer
  source 1..20: mol_type = other DNA
ccagatcaaggggaatgcta

SEQ ID NO: 122 - DNA, 20 bp, synthetic construct
  misc_feature 1..20: note = Primer
  source 1..20: mol_type = other DNA
caaggcagaccaatccattt

SEQ ID NO: 123 - DNA, 20 bp, synthetic construct
  misc_feature 1..20: note = Primer
  source 1..20: mol_type = other DNA
caaagtaagcatccccagga

SEQ ID NO: 124 - DNA, 20 bp, synthetic construct
  misc_feature 1..20: note = Primer
  source 1..20: mol_type = other DNA
gcagcacctctttcctcatc

SEQ ID NO: 125 - DNA, 20 bp, synthetic construct
  misc_feature 1..20: note = Primer
  source 1..20: mol_type = other DNA
tggaaattttgggctgtttc

SEQ ID NO: 126 - DNA, 20 bp, synthetic construct
  misc_feature 1..20: note = Primer
  source 1..20: mol_type = other DNA
tttctccagggaatttgtgc

SEQ ID NO: 127 - DNA, 20 bp, synthetic construct
  misc_feature 1..20: note = Primer
  source 1..20: mol_type = other DNA
gctgctggagaccagaaaag

SEQ ID NO: 128 - DNA, 20 bp, synthetic construct
  misc_feature 1..20: note = Primer
  source 1..20: mol_type = other DNA
cgaagggcacctattcaaaa

SEQ ID NO: 129 - DNA, 33 bp, Equus caballus
  source 1..33: mol_type = other DNA
  CDS 1..33: protein_id = 145; transl_table = 1; translation = AADRTSAEPES
gccgccgaccgaacttcggcagagcccgagagc

SEQ ID NO: 130 - protein, 11 aa, Equus caballus
AADRTSAEPES

SEQ ID NO: 131 - DNA, 33 bp, Equus caballus
  source 1..33: mol_type = other DNA
  CDS 1..18: protein_id = 146; transl_table = 1; translation = AADRT
gccgccgaccgaacttaggcagagcccgagagc

SEQ ID NO: 132 - protein, 5 aa, Equus caballus
AADRT

SEQ ID NO: 133 - protein, 83 aa, Bos taurus
ANRPPLEVLKKIFPHQKPAVLELILKGCGGDLVSAVEVLLSSRSSSSAADRTSAEPEGLVLPSNGHLFEHTLSSYPLSSSKWS

SEQ ID NO: 134 - protein, 83 aa, Equus caballus
ANRPPLEVLKKIFPNQKPTVLELILKGCGGDLVSAVEVLLSSRSSASAADRTSAEPESLVLPSNGHTFEHTLSSYPTSSSKWS

SEQ ID NO: 135 - protein, 83 aa, Equus caballus
ANRPPLEVLKKIFPNQKPTVLELILKGCGGDLVSAVEVLLSSRSSASAADRTXAEPESLVLPSNGHTFEHTLSSYPTSSSKWS

SEQ ID NO: 136 - protein, 83 aa, Homo sapiens
ANRPPLEVLKKIFPNQKPTVLELILKGCGGDLVSAVEVLLSSRSSVTGAERTSAEPESLALPSNGHIFEHTLSSYPISSSKWS

SEQ ID NO: 137 - protein, 83 aa, Pan troglodytes
ANRPPLEVLKKIFPSQKPTVLELILQGCGGDLVGAVEVLLSSRCSVTGAERSCAEPESLLLPPRGHIFEHTLSSYPISSSKWS

SEQ ID NO: 138 - protein, 84 aa, Canis sp.
  source 1..84: note = Canis familiaris
ANRPPLEVLKKIFPGQKPAVLELILKGCGGDLVGAVEVLLSSRSSAAAASERTAAEPEALVLPPNGHLFEHGLGSYPLSSSKWS

SEQ ID NO: 139 - protein, 81 aa, Mus musculus
ANRPPLEVLKKIFPNQKPTVLELILKGCGGDLVSAVEVLLSSRSSAAGTERTAEESLVLPSSGHIFEHTLGSYPISSSKWS

SEQ ID NO: 140 - protein, 81 aa, Rattus norvegicus
ANRPPLEVLKKIFPNQKPTVLELILKGCGGDLVSAVEVLLSSRSSAAGAERTAEESLVLPSSGHIFEHTLGSYPISSSKWS

SEQ ID NO: 141 - protein, 82 aa, Gallus gallus
ANRPPLEVLKKIFPNQKPTVLELILKGCGGDLVGAVEVLLSSRSSVAGGERTSESDGLVLPSNGHIFEHTLSSYPISSSKWS

SEQ ID NO: 142 - protein, 83 aa, Danio rerio
ANRPPLEVLKKIFPAHKPAVLELILKGCGGDLVGAIEILLSSRSTMKPEKILSESSDALVLPSNGHLFEHPLSSYPVSSSKWS

SEQ ID NO: 143 - protein, 474 aa, Equus caballus; identical to SEQ ID NO: 2

SEQ ID NO: 144 - protein, 300 aa, Equus caballus; identical to SEQ ID NO: 4

SEQ ID NO: 145 - protein, 11 aa, Equus caballus
AADRTSAEPES

SEQ ID NO: 146 - protein, 5 aa, Equus caballus
AADRT

(19) United States
(12) Patent Application Publication
(10) Pub. No.: US 2025/0122573 A1
(43) Pub. Date: Apr. 17, 2025

(54) METHOD TO PREDICT THE PATTERN OF LOCOMOTION IN HORSES

(71) Applicants: Lisa S. Andersson, UPPSALA (SE); Leif Andersson, UPPSALA (SE); Gabriella Lindgren, KNIVSTA (SE)

(72) Inventors: Lisa S. Andersson, UPPSALA (SE); Leif Andersson, UPPSALA (SE); Gabriella Lindgren, KNIVSTA (SE)

(73) Assignee: Capilet Genetics AB, Vasteras (SE)

(21) Appl. No.: 18/921,127

(22) Filed: Oct. 21, 2024

Related U.S. Application Data
(63) Continuation of application No. 17/523,482, filed on Nov. 10, 2021.
Application No. 17/523,482 is a continuation of application No. 16/447,027, filed on Jun. 20, 2019, now abandoned.
Application No. 16/447,027 is a continuation of application No. 15/682,124, filed on Aug. 21, 2017, now Pat. No. 10,378,057.
Application No. 15/682,124 is a continuation of application No. 14/795,415, filed on Jul. 9, 2015, now abandoned.
Application No. 14/795,415 is a continuation of application No. 13/696,128, filed on Nov. 5, 2012, now abandoned.
(60) Provisional application No. 61/514,749, filed on Aug. 3, 2011.

(30) Foreign Application Priority Data
May 5, 2011 (SE) 1130034-0

(51) Int. Cl. C12Q 1/6883; A01K 29/00; C12Q 1/6876; C12Q 1/6881; G01N 33/68

(52) U.S. Cl. CPC C12Q 1/6883; A01K 29/00; C12Q 1/6876; C12Q 1/6881; G01N 33/6872; G01N 33/6875; C12Q 2600/124; C12Q 2600/156; G01N 2333/4703

(57) ABSTRACT

The present invention provides methods for predicting the pattern of locomotion in a horse including the ability of a horse to use different gaits and the ability to trot at a fast speed. The methods comprise determining in a sample of DNA obtained from a horse the presence or absence of at least one genetic marker, wherein said at least one genetic marker is located on horse chromosome 23, said marker being associated with the ability to use different gaits. The invention further provides primers that amplify markers being associated with the ability to use different gaits and hybridization probes to detect markers being associated with the ability to use different gaits and the ability to trot at a fast speed.

### FIELD OF INVENTION

The present invention relates to methods for predicting the pattern of locomotion in horses including the ability of a horse to use different gaits and the ability to trot or pace at a fast speed. The methods comprise determining in a sample of DNA obtained from a horse the allele of at least one genetic marker, wherein said at least one genetic marker is located on horse chromosome 23, said marker being associated with the ability to use different gaits.

### BACKGROUND

Horses show a considerable variation in their pattern of locomotion both within and between breeds. The three basic gaits in horses are walk, trot and gallop. The horses use these different gaits according to their speed, walk is used at slow speed, trot is a faster mode of locomotion and gallop is the gait horses normally use to run fast. However, some horses have the ability to also use alternative gaits, for example pace and toelt, and such horses are called gaited horses. A horse that pace moves the two legs on the same side in a lateral movement in contrast to a trotting horse that makes a diagonal movement where the diagonal front and hind legs move forward and backwards together. Furthermore, Icelandic horses are able to perform a fifth gait named toelt, which is a four beet gait with the same foot fall pattern as the walk. A characteristic feature of toelt is that the horse then always has at least one hoof touching the ground, giving a very smooth gait. Examples of other similar alternative gaits, also known as ambling gaits, are fox trot, the rack, running walk and paso cort. The alternative gaits vary in footfall pattern, timing, and cadence, and can be generally divided into four categories: pace, regular rhythm ambling, lateral ambling and diagonal ambling. Table 1 provides a classification of breeds as gaited or non-gaited horses. Most horse breeds are in fact non-gaited and only representative examples of such breeds are listed in the table. Horses representing breeds classified as non-gaited never or rarely are able to perform the alternative gaits whereas most or all horses from the gaited breeds can perform alternative gaits. There are more gaited breeds worldwide in addition to the ones listed in table 1. Sometimes, there is a considerable variation also within breeds as regards the pattern of locomotion. For instance, Icelandic horses are classified as four-gaited or five-gaited, where the former can perform walk, trot, gallop and toelt whereas the latter can also pace.

The Standardbred horse, used for harness racing has a unique ability to trot or pace at a very fast speed without falling into gallop which is the normal gait at high speed for a horse. In North America, a subpopulation of Standardbred horses that pace at very high speed has been developed. Other horse breeds used for harness racing includes breeds like the Cold-blooded trotter, Finnhorses, the Frensch trotter and the Orlove trotter.

The pattern of locomotion in horses is under strong selection in horse breeding. For instance, the ability to race using gallop, trot and pace are selected in Thoroughbred horses, Standardbred trotters and Standardbred pacers, respectively. Horses with the ability to use alternative gaits are also highly desired by some riders and is a trait upon which many specialized breeds have been developed. Methods for predicting the pattern of locomotion in a horse, i.e. its ability to use different gaits, would therefore have a great utility in the horse breeding industry.

### BRIEF DESCRIPTION OF INVENTION

The Sequence Listing XML named 17186-90330-2215, created on 23 Dec. 2024 and of 179,524 bytes in size is incorporated herein by reference.

The present inventors have identified a genetic locus in horses that determines the horse's ability to use different gaits and the ability to trot at a fast speed. A premature stop codon in the gene for the doublesex and mab-3 related transcription factor 3 (DMRT3) was found in all tested horses with the ability to perform alternative gaits. Mutant horses express a truncated DMRT3 protein which lacks the last 174 amino acid residues but maintains a functional DNA-binding domain. DMRT3 is expressed in a subset of neurons in the spinal cord of the horse.

Accordingly the present invention provides methods for predicting the pattern of locomotion in horses including the ability of a horse to use different gaits, the ability to trot or pace at a fast speed, and the ability to perform in dressage.

A first aspect of the invention provides methods for predicting the pattern of locomotion in horses including the ability of a horse to use alternative gaits, the ability to trot at a fast speed, and the ability to perform in dressage which comprise extracting protein from a sample obtained from a horse. The methods further comprise determining in said protein sample the presence or absence of a truncated form of the DMRT3 protein. The DMRT3 protein can be a DMRT3 protein truncated at amino acid position 300 corresponding to the protein SEQ ID NO: 4. The determination can be made by use of an immunochemical method, such as Western blot, using an anti DMRT3 antibody.

A second aspect of the invention provides methods for predicting the pattern of locomotion in horses including the ability of a horse to use alternative gaits, the ability to trot at a fast speed, and the ability to perform in dressage which comprise extracting DNA from a sample obtained from a horse. The methods further comprise determining in said DNA the allele of at least one genetic marker, wherein said at least one genetic marker is located in the region between the flanking SNPs at nucleotide positions 22,628,976 (corresponding to position 51 in SEQ ID NO: 6) and 23,315,071 (corresponding to position 51 in SEQ ID NO: 7) on horse chromosome 23.

The genetic marker can be selected from single nucleotide polymorphisms (SNPs) and insertion/deletions (INDELs).

Preferably, the genetic marker is selected from the genetic markers listed in Tables 4, 5, 7 and 8.

Preferably the genetic marker is located in the region between the flanking SNPs at nucleotide positions 22,919,878 and 23,011,289 on horse chromosome 23.

Preferably, the genetic marker is selected from the genetic markers listed in Table 8.

Most preferably the genetic marker is located at position 22,999,655 on horse chromosome 23, corresponding to position 939 in SEQ ID NO:1.

More specifically, the methods can comprise identifying in said DNA the nucleotide in one or more specific position(s) selected from the positions 22,919,878; 22,920,361; 22,920,434; 22,920,646; 22,920,717; 22,921,203; 22,922,079; 22,922,780; 22,923,569; 22,924,120; 22,924,142; 22,924,299; 22,924,380; 22,924,407; 22,926,098; 22,926,188; 22,926,872; 22,927,387; 22,927,607; 22,928,220; 22,928,537; 22,928,587; 22,929,137; 22,930,011; 22,932,024; 22,932,895; 22,933,218; 22,936,034; 22,940,759; 22,942,423; 22,945,643; 22,946,599; 22,948,774; 22,949,055; 22,949,108; 22,949,240; 22,949,710; 22,956,846; 22,960,132; 22,960,528; 22,960,710; 22,964,042; 22,965,059; 22,967,119; 22,967,656; 22,967,915; 22,968,898; 22,973,984; 22,974,589; 22,979,124; 22,980,014; 22,982,879; 22,984,588; 22,985,746; 22,988,210; 22,988,991; 22,993,092; 22,994,591; 22,999,058; 22,999,655; 23,002,606; 23,003,956; 23,008,772; 23,008,789; 23,009,648; 23,010,164; and 23,011,289, on horse chromosome 23.

Most preferably the methods comprise identifying in said DNA the nucleotide in the specific position 22,999,655 on horse chromosome 23.

More specifically, the methods can comprise determining in said DNA the presence or absence of:
i) the nucleotide C in a nucleotide position corresponding to position 939 in SEQ ID NO: 1,
ii) the nucleotide A in a nucleotide position corresponding to position 939 in SEQ ID NO: 3,
iii) the nucleotide C and/or T in a nucleotide position corresponding to position 51 in SEQ ID NO: 5,
iv) the nucleotide A and/or G in a nucleotide position corresponding to position 51 in SEQ ID NO: 6,
v) the nucleotide C and/or T in a nucleotide position corresponding to position 51 in SEQ ID NO: 7,
vi) the nucleotide G and/or C in a nucleotide position corresponding to position 51 in SEQ ID NO: 8,
vii) the nucleotide A and/or G in a nucleotide position corresponding to position 51 in SEQ ID NO: 9,
viii) the nucleotide T and/or G in a nucleotide position corresponding to position 51 in SEQ ID NO: 10,
ix) the nucleotide T and/or C in a nucleotide position corresponding to position 51 in SEQ ID NO: 11,
x) the nucleotide C and/or T in a nucleotide position corresponding to position 51 in SEQ ID NO: 12,
xi) the nucleotide A and/or G in a nucleotide position corresponding to position 51 in SEQ ID NO: 13,
xii) the nucleotide A and/or C in a nucleotide position corresponding to position 51 in SEQ ID NO: 14
xiii) the nucleotide G and/or C in a nucleotide position corresponding to position 51 in SEQ ID NO: 15,
xiv) the nucleotide C and/or T in a nucleotide position corresponding to position 51 in SEQ ID NO: 16,
xv) the nucleotide G and/or A in a nucleotide position corresponding to position 51 in SEQ ID NO: 17,
xvi) the nucleotide G and/or C in a nucleotide position corresponding to position 51 in SEQ ID NO: 18,
xvii) the nucleotide C and/or A in a nucleotide position corresponding to position 51 in SEQ ID NO: 19,
xviii) the nucleotide T and/or C in a nucleotide position corresponding to position 51 in SEQ ID NO: 20,
xix) the nucleotide C and/or T in a nucleotide position corresponding to position 51 in SEQ ID NO: 21,
xx) the nucleotide C and/or T in a nucleotide position corresponding to position 51 in SEQ ID NO: 22,
xxi) the nucleotide C and/or A in a nucleotide position corresponding to position 51 in SEQ ID NO: 23,
xxii) the nucleotide C and/or G in a nucleotide position corresponding to position 51 in SEQ ID NO: 24,
xxiii) the nucleotide A and/or T in a nucleotide position corresponding to position 51 in SEQ ID NO: 25,

Preferably the methods comprise determining in said DNA the presence or absence of:
i) the nucleotide C in a nucleotide position corresponding to position 939 in SEQ ID NO: 1,
ii) the nucleotide A in a nucleotide position corresponding to position 939 in SEQ ID NO: 3,
iii) the nucleotide C and/or T in a nucleotide position corresponding to position 51 in SEQ ID NO: 5,
iv) the nucleotide C and/or T in a nucleotide position corresponding to position 51 in SEQ ID NO: 7,
v) the nucleotide T and/or C in a nucleotide position corresponding to position 51 in SEQ ID NO: 20,
vi) the nucleotide C and/or T in a nucleotide position corresponding to position 51 in SEQ ID NO: 21,
vii) the nucleotide C and/or T in a nucleotide position corresponding to position 51 in SEQ ID NO: 22,
viii) the nucleotide C and/or A in a nucleotide position corresponding to position 51 in SEQ ID NO: 23,
ix) the nucleotide C and/or G in a nucleotide position corresponding to position 51 in SEQ ID NO: 24,
x) the nucleotide A and/or T in a nucleotide position corresponding to position 51 in SEQ ID NO: 25,

Most preferably the methods comprise determining in said DNA the presence or absence of:
i) the nucleotide C in a nucleotide position corresponding to position 939 in SEQ ID NO: 1. or
ii) the nucleotide A in a nucleotide position corresponding to position 939 in SEQ ID NO: 3.

The horse can be selected from any horse or breed of horses belonging to the species Equus caballus. Examples of horse breeds can be found in Table 1.

TABLE 1
Classification of horse breeds as gaited or non-gaited, where
gaited horses have the ability to perform alternative gaits
in addition to the three basic gaits walk, trot and gallop.

| Breed | Classification |
|---|---|
| American Saddlebred | gaited |
| Campolina | gaited |
| Icelandic horse | gaited |
| Kentucky Mountain Saddle Horse | gaited |
| Mangalarga Marchador | gaited |
| Marwari horse | gaited |
| Missouri Foxtrotter | gaited |
| Paso Fino | gaited |
| Peruvian Paso | gaited |
| Racking horse | gaited |
| Rocky Mountain Horse | gaited |
| Spotted Saddle horse | gaited |
| Standardbred * | gaited |
| Tennessee Walker | gaited |
| Walkaloosa | gaited |
| Akhal teke | non-gaited |
| American Paint Horse | non-gaited |
| Andalusian | non-gaited |
| Arabian | non-gaited |
| Belgian | non-gaited |
| Dole | non-gaited |
| Exmoor Pony | non-gaited |
| Friesian | non-gaited |
| Haflinger | non-gaited |
| Hanoverian | non-gaited |
| Lusitano | non-gaited |
| North Swedish Draft horse | non-gaited |
| Norwegian Fjord | non-gaited |
| Quarter Horse | non-gaited |
| Selle Francais | non-gaited |
| Shetland Pony | non-gaited |
| Suffolk Punch | non-gaited |
| Thoroughbred | non-gaited |
| Trakehner | non-gaited |

* Two separate populations, pacers and trotters, many trotters seem to be able to toelt.

According to one aspect of the invention the methods according to the present invention can be used for paternity testing of horses.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.” These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### LEGENDS TO FIGURES

The following drawings form part of the present specification and are included to further demonstrate certain aspects of the present invention. The invention may be better understood by reference to one or more of these drawings in combination with the detailed description of specific embodiments presented herein.

FIG. 1. Results of genome-wide analysis of 70 Icelandic horses classified as four-gaited or five-gaited. The highly associated SNP at nucleotide position Chr23:22,967,656 base pairs is marked by an arrow.

FIG. 2. Genomic region harboring the Gait locus on chromosome 23 controlling the pattern of locomotion in horses. The DMRT3 gene is not properly annotated in this assembly but it is represented by the Ensembl transcript ENSECAT00000025062 indicated by an arrow in this figure. The figure is adapted from an output from the UCSC genome browser (www.genome.ucsc.edu).

FIG. 3. Nucleotide and amino acid alignment for codon 296 to 306 of horse DMRT3 including codon 301 in which a nonsense mutation occurs in the allele associated with the ability to pace.

FIG. 4. Alignment of amino acids 249 to 331 (numbered according to the horse sequence) in the DMRT3 protein from different vertebrate species including the wild-type (WT) and mutant (MUT) form of the horse DRMT3 protein. “.” indicates gap in the alignment; “-” indicates identity to the master sequence used (cattle); * indicates the nonsense mutation at codon 301 in the horse mutant allele.

FIG. 5. Schematic presentation of the predicted wild-type and mutant (gait) forms of the DMRT3 protein in horses. DM=zinc-finger like DNA binding module. DMA=protein domain of unknown function present in DMRT proteins.

FIG. 6. EMSA using an oligonucleotide representing a DMRT3-binding motif and in vitro-translated myc-tagged DMRT3 wild-type and mutant proteins.

Super-shifts were demonstrated using an anti-myc antibody (that recognizes both forms) or with an anti-DMRT3 antibody that recognizes the C-terminal part of the wild-type protein, but not the truncated form. An oligonucleotide corresponding to a DMRT1-binding site was also used and gave similar results (data not shown). The cold competing oligonucleotide was added in 150X excess. GS=gel-shift representing complex between DMRT3 protein and oligonucleotide; SS=super-shift representing complex between antibody, DMRT3 protein and oligonucleotide.

### DETAILED DESCRIPTION OF THE INVENTION

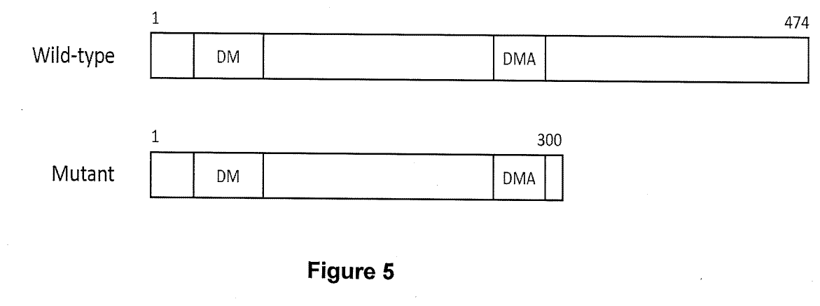

The present inventors have demonstrated that there is a locus, here named Gait, on horse chromosome 23 that has a major impact on the pattern of locomotion in horses. The present results show that homozygosity for a recessive allele at this locus is required for the ability of a horse to pace. It is postulated that the nonsense mutation at nucleotide position 22,999,655 in exon 2 of the DMRT3 gene is the causative mutation for the Gait allele. DMRT3 is a highly conserved gene present in all vertebrates studied so far. The function of the DMRT3 protein has not been established by any previous studies but the fact that it is expressed in brain and in the spinal cord of the mouse (MGI, www.informatics.jax.org) is consistent with a critical role for controlling locomotion as demonstrated by the present study. The nonsense mutation underlying the gait allele may very well have a phenotypic effect in the heterozygous condition since it occurs in the last exon of DMRT3 and is expected to encode a truncated form of the protein (SEQ ID NO:4) that lacks the last 174 amino acids (FIG. 5). The DNA binding DM domain of DMRT3 is located in the N-terminal part that is maintained in the truncated form (FIG. 5). The mutant form of DMRT3 may therefore be able to bind to its target DNA sequences but may show defects as regards the interaction with other proteins required for its normal function and may therefore has a dominant-negative effect in heterozygotes. It is worth noticing that only one of the Icelandic horses was homozygous for the wild-type (non-pace) allele at the Gait locus.

This study has established a genetic marker that can be used to predict the genetic constitution of a horse as regards its pattern of locomotion. We predict that the gait allele is present in most, if not all, gaited breeds some of which are listed as gaited in Table 1 and it may occur at a low frequency in other breeds as well. The marker also predicts a horse capacity to trot or pace at a high speed as it is found at a high frequency in horses used for harness racing. Further, we predict that horses with at least one wild-type allel are better at showjumping, traditional dressage, and completion racing in gallop.

The pattern of locomotion determines the ability of a horse to use alternative gaits, as well as the horse's ability to trot or pace at a fast speed, and its ability to perform in dressage. Alternative gaits include, pace, and the ambling gaits exemplified by toelt, running walk, rack, classic fino, paso corto, paso largo, paso ilano, sobreandando, fox trot.

A horse being homozygous or heterozygous for the gait allele can be predicted to have the ability to use alternative gaits and to trot at high speed. A horse being homozygous or heterozygote for the wild type allele can be predicted to have better ability to perform in showjumping, dressage, and completion racing in gallop.

The utility of this invention in the horse breeding industry includes the determination of the genotype of potential breeding animals to maximise the chance to obtain a progeny with a favoured pattern of locomotion. The information about the genotype at the DMRT3 locus may also be used by sellers and buyers of horses to predict the ability of the horse to perform different gaits. Furthermore, the methods according to the invention can be used to effectively introgress the gait allele into non-gaited breeds.

According to one aspect of the invention the methods according to the present invention can be used for selecting horses for breeding.

Accordingly, one aspect of the invention provides methods for selecting a horse for breeding, said methods comprising determining in a DNA sample obtained from said horse the allele of at least one genetic marker, wherein said at least one genetic marker is located in the region between the flanking SNPs at nucleotide positions 22,628,976 on horse chromosome 23. The genetic marker can be selected from single nucleotide polymorphisms (SNPs) and insertion/deletions (INDELs).

Preferably, the genetic marker is selected from the genetic markers listed in Tables 4, 5, 7 and 8.

Preferably the genetic marker is located in the region between the flanking SNPs at nucleotide positions 22,919,878 and 23,011,289 on horse chromosome 23.

Preferably, the genetic marker is selected from the genetic markers listed in Table 8.

Most preferably the genetic marker is located at position 22,999,655 on horse chromosome 23, corresponding to position 939 in SEQ ID NO:1.

The most reliable test for determining the genotype at the Gait locus is to determine the presence and/or absence of the nonsense mutation in exon 2 of DMRT3 (nucleotide position 22,999,655 on chromosome 23, corresponding to nucleotide position 939 in SEQ ID NO:3). However, genetic markers located in the interval between the flanking markers at nucleotide positions 22,628,976 and 23,315,071, and more specifically genetic markers located in the interval between positions 22,919,878 and 23,011,289, exemplified by the markers listed in Table 8, show a more or less strong association to the genotype for the causative SNP at nucleotide position 22,999,655 due to the presence of linkage disequilibrium in the region. Accordingly, one or more of these markers, individually or in combination, can be used to determine the genotype at the Gait locus, and can consequently as well be used in the methods according to the present invention.

The term “sample” or “biological sample” according to the present invention refers to any material containing nucleated cells from said horse to be tested. In a preferred embodiment the biological sample to be used in the methods of the present invention is selected from the group consisting of blood, sperm, hair roots, milk, body fluids as well as tissues including nucleated cells.

DNA extraction, isolation and purification methods are well-known in the art and can be applied in the present invention. Standard protocols for the isolation of genomic DNA are inter alia referred to in Sambrook, J., Russell. D. W., Molecular Cloning: A Laboratory Manual, the third edition, Cold Spring Harbor Laboratory Press, Cold Spring Harbor. New York, 1.31-1.38, 2001 and Sharma. R.C., et al. “A rapid procedure for isolation of RNA-free genomic DNA from mammalian cells”, BioTechniques, 14. 176-178. 1993.

According to the present invention the term “SNP” refers to a single nucleotide polymorphism at a particular position in the horse genome that varies among a population of individuals. SNPs can be identified by their location within the disclosed particular sequence, i.e. within the interval of 22,628,976 and 23,315,071 base pairs on horse chromosome 23 or their name as shown in Tables 4, 5, 7 and 8. SNPs identified as being useful for predicting the ability of a horse to use different gaits according to the present invention are shown in Tables 4, 5, 7 and 8. For example, the SNP BIEC2-620109 of Table 5 indicates that the nucleotide base (or the allele) at nucleotide position 22,967,656 on chromosome 23 of the reference sequence as referred to herein may be either Cytosine (C) or Thymidine (T). The allele associated with or indicative for a horse able to use five gaits is in the case of SNP BIEC2-620109 of Table 5 Thymidine (T).

The term “determining in said DNA the allele of at least one genetic marker” in accordance with the present invention refers to a method for determining or identifying whether a particular nucleotide sequence is present in a DNA sample.

The term “identifying in said DNA the nucleotide in one or more specific position on the horse chromosome 23” refers to a method for determining the identity of the nucleotide in said specific position on the horse chromosome 23, i.e. to determine whether the nucleotide in said specific position is Adenosine (A), Cytosine (C), Guanosine (G), or Thymidine (T).

There are several methods known by those skilled in the art for determining whether a particular nucleotide sequence is present in a DNA sample and for identifying the nucleotide in a specific position in a DNA sequence. These include the amplification of a DNA segment encompassing the genetic marker by means of the polymerase chain reaction (PCR) or any other amplification method, interrogate the genetic marker by means of allele specific hybridization, the 3′exonuclease assay (Taqman assay), fluorescent dye and quenching agent-based PCR assay, the use of allele-specific restriction enzymes (RFLP-based techniques), direct sequencing, the oligonucleotide ligation assay (OLA), pyrosequencing, the invader assay, minisequencing, DHPLC-based techniques, single strand conformational polymorphism (SSCP), allele-specific PCR, denaturating gradient gel electrophoresis (DGGE), temperature gradient gel electrophoresis (TGGE), chemical mismatch cleavage (CMC), heteroduplex analysis based system, techniques based on mass spectroscopy, invasive cleavage assay, polymorphism ratio sequencing (PRS), microarrays, a rolling circle extension assay, HPLC-based techniques, extension based assays, ARMS (Amplification Refractory Mutation System), ALEX (Amplification Refractory Mutation Linear Extension), SBCE (Single base chain extension), molecular beacon assays, invader (Third wave technologies), ligase chain reaction assays, 5′-nuclease assay-based techniques, hybridization capillary array electrophoresis (CAE), protein truncation assays (PTT), immunoassays, and solid phase hybridization (dot blot, reverse dot blot, chips). This list of methods is not meant to be exclusive, but just to illustrate the diversity of available methods. Some of these methods can be performed in accordance with the methods of the present invention in microarray format (microchips) or on beads.

The invention thus also relates to the use of primers or primer pairs, wherein the primers or primer pairs hybridize(s) under stringent conditions to the DNA comprising the interval between nucleotide positions 22,628,976 and 23,315,071, preferably between positions 22,919,878 and 23,011,289, base pairs on horse chromosome 23, or to the complementary strand thereof.

Preferably the primers or primer pairs hybridize(s) under stringent conditions to the sequences SEQ ID NO: 1, 3 and 5 to 25.

Preferably, the primers of the invention have a length of at least 14 nucleotides such as 17 or 21 nucleotides.

More specifically the primers can be selected from SEQ NO:26, SEQ ID NO:27, SEQ ID NO:30, and SEQ ID NO:31.

In one embodiment, the primers actually binds to the position of the SNPs as referred to in Tables 4, 5, 7 and 8. Such an allele specific oligonucleotide in accordance with the present invention is typically an oligonucleotide of at least 14 to 21 nucleotide bases in length designed to detect a difference of a single base in the target's genetic sequence of the horse to be tested. In accordance with the present invention one or more specific primers can be applied in order to identify more than a single SNP as referred to herein. As a consequence, when binding is performed under stringent conditions, such primer or such primers is/are useful to distinguish between different polymorphic variants as binding only occurs if the sequences of the primer and the target have full complementarily. It is further preferred that the primers have a maximum length of 24 nucleotides. Such primers can be coupled with an appropriate detection method such as an elongation reaction or an amplification reaction, which may be used to differentiate between the polymorphic variants and then draw conclusions with regard to the horse as regards its ability to use different gaits.

Hybridisation is preferably performed under stringent or highly stringent conditions. “Stringent or highly stringent conditions” of hybridization are well known to or can be established by the person skilled in the art according to conventional protocols. Appropriate stringent conditions for each sequence may be established on the basis of well-known parameters such as temperature, composition of the nucleic acid molecules, salt conditions etc.: see, for example, Sambrook et al. “Molecular Cloning, A Laboratory Manual”, CSH Press, Cold Spring Harbor, 1989 or Higgins and Hames (eds.), “Nucleic acid hybridization, a practical approach”, IRL Press, Oxford 1985, see in particular the chapter “Hybridization Strategy” by Britten & Davidson. Typical (highly stringent) conditions comprise hybridization at 65° C. in 0.5×SSC and 0.1% SDS or hybridization at 42° C. in 50% formamide, 4×SSC and 0.1% SDS. Hybridization is usually followed by washing to remove unspecific signals. Washing conditions include conditions such as 65° C., 0.2×SSC and 0.1% SDS or 2×SSC and 0.1% SDS or 0.3×SSC and 0.1% SDS at 25° C.-65° C.

The term “nucleotide positions 22,628,976 and 23,315,071 base pairs on horse chromosome 23” and other similar denoted nucleotide positions refer to the horse reference sequence according to the September 2007 Equus caballus draft assembly EquCab2 (UCSC version equCab2). EquCab2 was produced by The Broad Institute. EquCab2 is available at the www.genome.ucsc.edu genome browser.

### EXAMPLES

A genome-wide screen for genes affecting pattern of locomotion using the horse SNP chip comprising assays for 54,602 single nucleotide polymorphisms in the horse genome (Illumina EquineSNP50 BeadChip; http://www.illumina.com/products/equine_snp50_whole_genome_genotyping_kits.ilmn) was performed. A population material comprising 70 Icelandic horses in which 30 were classified as four-gaited and 40 were classified as five-gaited, i.e. only the latter had a documented ability to pace, was used in the assay.

Animal material. Blood samples were collected from 70 Icelandic horses from Sweden. Genomic DNA was prepared from all horses using QIAamp DNA Blood Midi Kit (Qiagen). The owners of the horses were asked to classify their horses as four-gaited or five-gaited. Hair samples were collected from 61 Swedish Standardbred horses and 2 North-Swedish Trotter. DNA from six hair roots was extracted by adding 97 μl Chelex solution and 7 μl Proteinas K and incubated in 56° C. for 60 minutes followed by an incubation in 95° C. for 10 minutes.

Genome-wide analysis (GWA). The GWA was performed using the Illumina EquineSNP50 BeadChip (http://www.illumina.com/products/equine_snp50_whole_genome_genotyping_kits.ilmn). The statistical analysis of the data was carried out using the software PLINK (Purcell et al. 2007. PLINK: a tool set for whole-genome association and population-based linkage analyses. Am. J. Hum. Genet. 81:559-575).

DNA sequencing. A number of coding and non-coding regions located between the flanking SNPs at nucleotide positions 22,628,976 and 23,315,071 base pairs on horse chromosome 23 was PCR amplified and sequenced to identify sequence polymorphisms. All primers used for these experiments are listed in Table 2. The amplicons were amplified with standard PCR conditions and (2720 Thermal Cycler, Applied Biosystems, Foster City, CA). Standard Sanger sequencing was performed using an AB3730 capillary sequencer (Applied Biosystems, Foster City, CA).

TABLE 2
Primers used for PCR amplification and
sequencing of selected regions in horses

| | Amplified | | | | |
|---|---|---|---|---|---|
| | region | | | | |
| | Nucleo- | | SEQ | | SEQ |
| | tide | Forward | ID | Reverse | ID |
| Amplicon | positions | primer | NO: | primer | NO: |
| ANKRD15 | chr23: | TCATACCAGCT | 35 | GAGGAGAGAGA | 36 |
| exon1.1 | 22792627- | TGCCACACT | | GCTCGTGGA | |
| | 22793280 | | | | |
| ANKRD15 | chr23: | CTAATGGAGAC | 37 | GCCGGAACTCC | 38 |
| exon1.2 | 22793162- | CCGCAGAAG | | TTTATCCTC | |
| | 22793792 | | | | |
| ANKRD15 | chr23: | GAGAAGTGGCG | 39 | GCCCCACGACT | 40 |
| exon1.3 | 22793704- | GGGAATTAT | | TTATTCTCA | |
| | 22794386 | | | | |
| ANKRD15 | chr23: | TGCAGACGAGA | 41 | AAACCCAGAAG | 42 |
| exon1.4 | 22794261- | GACCAAATG | | TGCCTGAGA | |
| | 22794946 | | | | |
| ANKRD15 | chr23: | GCGGACAGTGG | 43 | AATACATTGTC | 44 |
| exon1.5 | 22794844- | CTATAGGAG | | CCCACCCTTC | |
| | 22795453 | | | | |
| ANKRD15 | chr23: | ATGGGATTTGA | 45 | AAGCCTGATGC | 46 |
| exon2 | 22807940- | GCTGAGTGG | | TGAGAAGGA | |
| | 22808575 | | | | |
| ANKRD15 | chr23: | TTGCATGCACA | 47 | CTGGGGGTTTC | 48 |
| exon3 | 22809005- | CAATTTTCC | | TGAGTTCTG | |
| | 22809616 | | | | |
| ANKRD15 | chr23: | GCAACCCAGGT | 49 | TCACCTTCTGC | 50 |
| exon4 | 22810246- | TATCCCTTT | | ACTTGCATT | |
| | 22810904 | | | | |
| ANKRD15 | chr23: | AAGTCGACTGA | 51 | ACCTTGGCCCA | 52 |
| exon5 | 22812005- | GGGGCTCTT | | GATAGGTTT | |
| | 22812621 | | | | |
| ANKRD15 | chr23: | TCCCCAGGAAC | 53 | TGGAAAGGATT | 54 |
| exon6 | 22815102- | ATACAGCTC | | TGAGGATGC | |
| | 22815741 | | | | |
| ANKRD15 | chr23: | GCTTCTGGCCT | 55 | TGGCATGAAGA | 56 |
| exon7 | 22817755- | CACGAAATA | | CACCACAAT | |
| | 22818429 | | | | |
| ANKRD15 | chr23: | AGCCCCAGTAC | 57 | GGGAAGTCGCC | 58 |
| exon8 | 22818653- | AGACCACAC | | TACACTGAA | |
| | 22819254 | | | | |
| ANKRD15 | chr23: | GAGGATCCGTG | 59 | AGCAAGTCTCC | 60 |
| exon9 | 22820739- | GGATACAGA | | TGAGCAAGC | |
| | 22821346 | | | | |
| ANKRD15 | chr23: | CAGAGGACACA | 61 | CAAAACCATCC | 62 |
| exon10 | 22821626- | TCTGCCTGA | | TGGAAATGG | |
| | 22822233 | | | | |
| ANKRD_ | chr23: | GTCCATCCCCT | 63 | TGTCAGCTGCA | 64 |
| GAP | 22836558- | TCTCTCCTC | | GAATGGAAG | |
| | 22837273 | | | | |
| PRIMER_ | chr23: | AGACTGGCCCT | 65 | CTGAAGGTGCC | 66 |
| DS7 | 22851938- | GAGCTAACA | | CTCTACAGC | |
| | 22852292 | | | | |
| PRIMER_ | chr23: | TTACCTGCCCC | 67 | CATCTTTGCCC | 68 |
| DS5 | 22868140- | TTTGTTTTG | | CTCAGACTC | |
| | 22868803 | | | | |
| PRIMER_ | chr23: | TTACGTGGCAC | 69 | AGCCTGGACTC | 70 |
| DS2 | 22869516- | CCCTACTTC | | TGTCCTTGA | |
| | 22870124 | | | | |
| PRIMER_ | chr23: | TGCTGCCCTCT | 71 | AAAGTAACGAT | 72 |
| DS1 | 22872699- | GTCTATGTG | | GCGGTGGAC | |
| | 22873368 | | | | |
| PRIMER_ | chr23: | AAATGGCTGTG | 73 | CTGTGTGACCA | 74 |
| DS4 | 22874773- | CCGTTTTAC | | AGCTCTCCA | |
| | 22875445 | | | | |
| PRIMER_ | chr23: | GAAAATGCTGA | 75 | CTTGCTGCCTT | 76 |
| DS3 | 22876084- | CGTGCTGAA | | TTGCCTATC | |
| | 22876784 | | | | |
| PRIMER_ | chr23: | GCAGAGCGACC | 77 | GGCCTTAGAGG | 78 |
| DS6 | 22876563- | TGGAGATAG | | GACACATGA | |
| | 22877255 | | | | |
| BIETOP- | chr23: | CCTCTCACCCA | 79 | AGTTGGCAAAC | 80 |
| 620109B_ | 22967269- | GACACCATT | | AACAGGACA | |
| 3 | 22967902 | | | | |
| BIETOP- | chr23: | AAGTCCTTTCT | 81 | GGTCCATCGTT | 82 |
| 620109D_ | 22967525- | TGGGGGCTA | | GACCAAAAT | |
| 2 | 22968019 | | | | |
| BIETOP- | chr23: | AGTCCTTTCTT | 83 | ACGGCACCACC | 84 |
| 620109C_ | 22967526- | GGGGGCTAA | | ATCATCTAT | |
| 2 | 22968041 | | | | |
| DMRT3 | chr23: | GCCCCAACTTA | 85 | CCGCGCTGCTT | 86 |
| exon0 | 22985884- | AGACCCTCT | | AGGAGTC | |
| | 22986463 | | | | |
| DMRT3 | chr23: | GCCCCAACTTA | 87 | TACCTGGCTTG | 88 |
| exon0B | 22985884- | AGACCCTCT | | TCGAGCTG | |
| | 22987295 | | | | |
| DMRT3 | chr23: | GAGCACGCTCA | 89 | AAAGAGCTCCG | 90 |
| GAP | 22986413- | GACCCTATC | | AAGTTTTTGC | |
| | 22987358 | | | | |
| DMRT3 | chr23: | CTCCTTCCAAG | 91 | AGAGTCTGCGG | 92 |
| exon2.1 | 22999117- | AAGCCTGTG | | AAAACCTCA | |
| | 22999797 | | | | |
| DMRT3 | chr23: | CCTTGAGCTCA | 93 | ACTAAAGCCGC | 94 |
| exon2.2 | 22999709- | TACCCCATC | | AGAGCAGAG | |
| | 23000396 | | | | |
| DMRT3 | chr23: | GAGAGGCCTCG | 95 | TCCCACTCACA | 96 |
| exon2.3 | 23000251- | TCCTGTGTA | | TTTCCCAAT | |
| | 23001049 | | | | |
| PRIMER_1 | chr23: | CAAGGGCATGA | 97 | ACTCCATGATT | 98 |
| | 23009567- | GGAGTGTTT | | GCACAACGA | |
| | 23010210 | | | | |
| PRIMER_2 | chr23: | TCATTCCACCA | 99 | GGCCACTGCAG | 100 |
| | 23027620- | GCAATGTGT | | AAGAAAGAG | |
| | 23028300 | | | | |
| PRIMER_3 | chr23: | CTGTTGTCCCA | 101 | AGGTGAGTCCA | 102 |
| | 23048139- | GCCCTGTAT | | GGCTAGCAA | |
| | 23048767 | | | | |
| DMRT2 | chr23: | GAGCCCGAGCG | 103 | ATTAGGACCGC | 104 |
| exon1 | 23055803- | GATAATACT | | ACAGGACAC | |
| | 23056469 | | | | |
| DMRT2 | chr23: | GCGGCTAGGGT | 105 | CTCGTCCTCGT | 106 |
| exon2 | 23056584- | GGTACTTCT | | CCTCGTC | |
| | 23057237 | | | | |
| DMRT2_ | chr23: | GAGGACGACGA | 107 | CCACTTTCAAG | 108 |
| GAP | 23057214- | GGACGAG | | GCCTCTCTG | |
| | 23057971 | | | | |
| DMRT2 | chr23: | GAGGACGACGA | 109 | CCACTTTCAAG | 110 |
| exon2GAP | 23057214- | GGACGAG | | GCCTCTCTG | |
| | 23057971 | | | | |
| DMRT2 | chr23: | CTGGGGTGACT | 111 | TCACACCAAGG | 112 |
| exon3 | 23059113- | CTAGCAAGG | | CAAATTTCA | |
| | 23059736 | | | | |
| DMRT2 | chr23: | CCCCCAAAGGG | 113 | GAACTGAGGTG | 114 |
| exon4.1 | 23061639- | AACTATTTT | | GTGGCATTT | |
| | 23062293 | | | | |
| DMRT2 | chr23: | TTCAGGGTCTG | 115 | TCCAACTTGTT | 116 |
| exon4.2 | 23062130- | GGAATATGG | | TGGCTACGA | |
| | 23062788 | | | | |
| DMRT2 | chr23: | GGCCCCTAAGA | 117 | CCTGTAGACCC | 118 |
| exon4.3 | 23062686- | AACACAGAG | | CAGAGACCA | |
| | 23063285 | | | | |
| PRIMER_4 | chr23: | GGTCCAAATTG | 119 | TTCCCCAGGAG | 120 |
| | 23067103- | TAGGGCTGA | | GTTCTCTTT | |
| | 23067766 | | | | |
| PRIMER_5 | chr23: | CCAGATCAAGG | 121 | CAAGGCAGACC | 122 |
| | 23069404- | GGAATGCTA | | AATCCATTT | |
| | 23070095 | | | | |
| PRIMER_6 | chr23: | CAAAGTAAGCA | 123 | GCAGCACCTCT | 124 |
| | 23076510- | TCCCCAGGA | | TTCCTCATC | |
| | 23077194 | | | | |
| PRIMER_7 | chr23: | TGGAAATTTTG | 125 | TTTCTCCAGGG | 126 |
| | 23080154- | GGCTGTTTC | | AATTTGTGC | |
| | 23080820 | | | | |
| PRIMER_8 | chr23: | GCTGCTGGAGA | 127 | CGAAGGGCACC | 128 |
| | 23085336- | CCAGAAAAG | | TATTCAAAA | |
| | 23086005 | | | | |

In depth genome resequencing. DNA samples from two Icelandic horses, one female mutant DMRT3 homozygote and one male control (homozygous wild-type) were prepared for sequencing. Illumina paired-end libraries were generated from these DNA samples (mean insert sizes of approximately 220 bases). The two libraries were sequenced (2×100 bp) on seven and five lanes, respectively, using an Illumina HiSeq instrument. The reads were mapped to the horse genome (EquCab2 reference assembly) using the software BWA, and PCR-duplicates were removed using the software Picard (http://picard.sourceforge.net). The average read depth obtained for each sample was approximately 30×. SNPs and small insertions/deletions were called from the mapping data after subjecting the alignments to realignment around indels and then variant calling using the Genome Analysis Toolkit (GATK). The variant calls were subjected to recommended VariantFiltration Walker filters for SNPs listed in the GATK wiki page (http://www.broadinstitute.org/gsa/wiki/index.php/The Genome Analysis Toolkit) and read alignments overlapping SNP and insertion/deletion calls within the 438 kb Gait locus were then manually reviewed to remove obvious artifact calls. Read depths observed in one kilobase windows were used to call candidate duplications in the minimum IBD region, and mapping distances and orientations between paired reads were used to detect structural variations in relation to the reference assembly. The software ANNOVAR was used to annotate SNPs in relation to Ensembl genes.

SNP analysis using TaqMan assays. TaqMan assays were designed to screen the SNPs at chromosome 23, nucleotide position 22,967,656 (BIEC2_620109; the SNP included in the Illumina SNP panel showing the strongest association to the phenotype) and at nucleotide position 22,999,655 (DMRT3.3; the SNP causing a premature Stop codon in DMRT3 exon 2). Custom TaqMan SNP Genotyping assays (Applied Biosystems, Foster City, CA) designed for these two SNPs are summarized in Table 3. Probe and primer designs were obtained from the Applied Biosystems web page (http://www5.appliedbiosystems.com/tools/cadt/) using the custom genotyping assays order option. The ABI PRISM 7900 HT sequence detection system for 384-well format (Applied Biosystems, Foster City, CA) was used for the analysis.

TABLE 3
Description of TaqMan assays for SNPs
at nucleotide positions 22,967,656 (BIEC2_620109)
and 22,999,655 (DMRT3.3) on horse chromosome 23.

| | | SEQ |
|---|---|---|
| | | ID |
| | | NO |
| BIEC2_620109 | | |
| Forward Primer | GCAAAGTGCAGAAATAGTCTTTTGGA | 26 |
| Seq. | | |
| Reverse Primer | CACTCTTTTGGAATGGTTCACATTAAGG | 27 |
| Seq. | | |
| Reference | C | |
| allele* | | |
| Reporter | TAGTGCAAACGGTACGTT | 28 |
| Sequence (FAM) | | |
| Non-reference | T | |
| allele | | |
| Reporter | AAATAGTGCAAACAGTACGTT | 29 |
| Sequence (VIC) | | |
| DMRT3.3 | | |
| Forward Primer | CCTCTCCAGCCGCTCCT | 30 |
| Seq. | | |
| Reverse Primer | TCAAAGATGTGCCCGTTGGA | 31 |
| Seq. | | |
| Reference | C | |
| allele* | | |
| Reporter | CTGCCGAAGTTCG | 32 |
| Sequence (VIC) | | |
| Non-reference | A | |
| allele | | |
| Reporter | CTCTGCCTAAGTTCG | 33 |
| Sequence (FAM) | | |

*according to the EquCab2 assembly (available at www.genome.ucsc.edu genome browser)

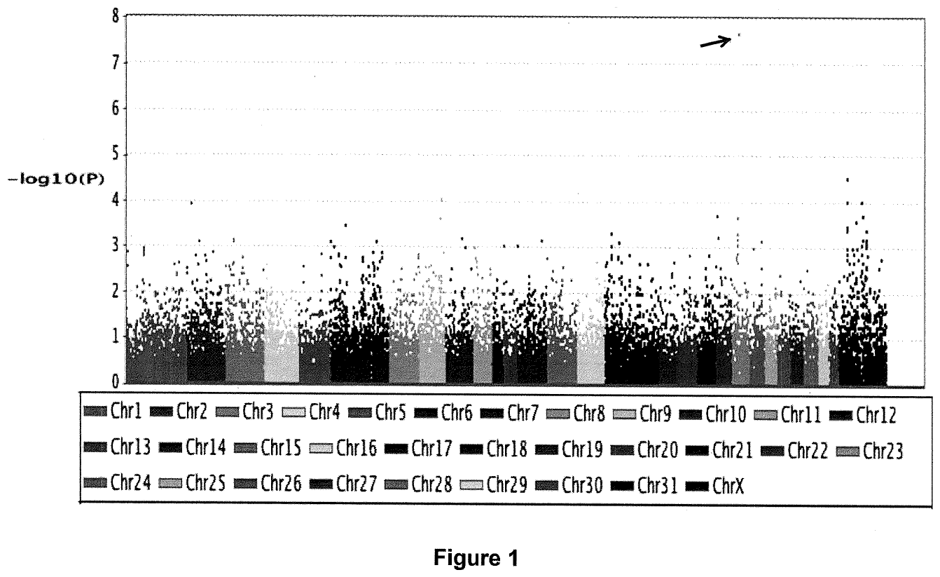

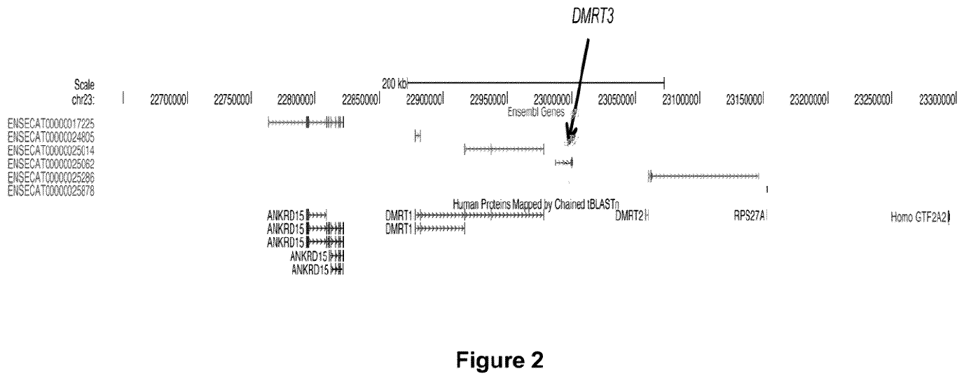

Genome-wide analysis reveals a locus on horse chromosome 23 controlling the pattern of locomotion. Statistical analysis of the SNP-chip data for the 70 Icelandic horses with a phenotypic classification as four-gaited or five-gaited was carried using PLINK; 39,695 SNPs passed the quality control. A chi-square test was performed for each marker separately in order to test for a significant difference in genotype frequencies between four-gaited versus five-gaited horses. A genetic model assuming a recessive mode of inheritance was used. Ten thousand permutations were used to correct for multiple testing. The statistical analysis revealed a highly significant association between a SNP (BIEC2_620109, SEQ ID NO: 5) at nucleotide position 22,967,656 base pair on horse chromosome 23 and the gait phenotype (P=0.0002, genome-wide significance; FIG. 1). The two SNPs immediately flanking the highly associated SNP were located at nucleotide positions 22,628,976 (BIEC2-619907, SEQ ID NO: 6) and 23,315,071 (BIEC2-620244, SEQ ID NO: 7) and these showed only weak associations to the phenotype (P=0.01 for the SNP at position 22,628,976 base pair and P=0.32 for the SNP at position 23,315,071 base pair). This result demonstrated that one or more sequence polymorphisms controlling the pattern of locomotion is located in the vicinity of the SNP at position 22,967,656 base pair (the most associated SNP) and within the interval defined by the flanking markers at positions 22,628,976 and 23,315,071 base pairs showing a significantly weaker association to the gait phenotype. This region spans 686 kilo base pairs and five genes are located in the interval ANKRD15, DMRT1, DMRT3, DMRT2 and GTF2A2 (FIG. 2). This locus was named the Gait locus and the results were consistent with a recessive inheritance of the allele associated with the ability to pace, while the wild-type allele (Non-pace) at this locus was dominant.

Resequencing of selected regions refine the localization of the Gait locus. A number of amplicons (Table 2) from the genomic region harbouring the Gait locus as defined by the genome-wide screen (from nucleotide position 22,628,976 to position 23,315,071 on chromosome 23) were resequenced in a small set of four-gaited and five-gaited horses in order to refine the localization of the Gait locus. All the sequence polymorphisms detected in this analysis are summarized in Table 4. The results showed that there is a distinct haplotype associated with the recessive gait allele and that the haplotype block showing a complete association to gait in this breed breaks up at nucleotide position 22,877,015 just upstream of the DMRT1 gene. The results refine the localization of the Gait locus to the interval from nucleotide position 22,877,015 base pair to position 23,315,071 base pair; ANKRD15 is located outside the critical interval for Gait.

TABLE 4
Sequence polymorphisms detected by resequencing amplicons from the
genomic region harbouring the Gait locus on horse chromosome 23

| | | Phenotype | | | | | | | | | | |
|---|---|---|---|---|---|---|---|---|---|---|---|---|
| | | Four-gaited | | | | Five-gaited | | | | | | |
| | | Horse | Horse | Horse | Horse | Horse | Horse | Horse | Horse | Horse | Horse | Horse |
| SNP | Position | 1 | 2 | 3 | 4 | 5 | 6 | 7 | 8 | 9 | 10 | 11 |
| ANKRD15.1 | 22,793,939 | GG | GC | GC | GG | GG | GG | GG | GG | GG | GG | GG |
| ANKRD15.2 | 22,810,322 | GG | GA | GA | GG | GA | GG | GA | GG | GG | GG | GA |
| ANKRD15.3 | 22,812,345 | GG | GT | GT | GG | GT | GG | GT | GG | GG | GG | GT |
| ANKRD15.4 | 22,812,251 | TT | TT | TT | TT | TC | TT | TC | TT | TT | TT | TC |
| ANKRD15.5 | 22,818,132 | TT | CT | CT | TT | CT | TT | CT | TT | TT | TT | CT |
| ANKRD15.6 | 22,818,158 | GG | GA | GA | GG | GA | GG | GA | GG | GG | GG | GA |
| ANKRD15.7 | 22,821,872 | CC | CA | CA | CC | CA | CC | CA | CC | CC | CC | CA |
| ANKRD15.8 | 22,821,884 | GG | GG | GG | GG | CG | GG | CG | GG | GG | GG | CG |
| SNP.1 | 22,868,190 | nt | nt | CC | CC | nt | nt | nt | nt | CC | CC | CT |
| SNP.2 | 22,868,678 | nt | nt | GA | AA | nt | nt | nt | nt | AA | AA | GA |
| SNP.3 | 22,872,820 | nt | nt | GG | GG | nt | nt | nt | nt | GG | GG | GC |
| SNP.4 | 22,876,848 | nt | nt | CA | AA | nt | nt | nt | nt | AA | AA | AA |
| SNP.5 | 22,877,015 | nt | nt | TT | TT | nt | nt | nt | nt | TT | TT | CT |
| BIEC2_620109 | 22,967,656 | CC | CC | CC | CT | TT | TT | TT | TT | TT | TT | TT |
| DMRT3.1 | 22,986,593 | TT | TT | TT | CT | CC | CC | nt | nt | CC | CC | CC |
| DMRT3.2 | 22,987,143 | CC | CC | CC | CT | TT | TT | nt | nt | TT | TT | TT |
| DMRT3.3 | 22,999,655 | CC | CC | CC | CA | AA | AA | nt | nt | AA | AA | AA |
| DMRT3.4 | 22,999,665 | GC | GG | GG | GC | CC | CC | nt | nt | CC | CC | CC |
| SNP.6 | 23,009,648 | nt | nt | AA | AT | nt | nt | nt | nt | TT | TT | TT |

nt = not tested

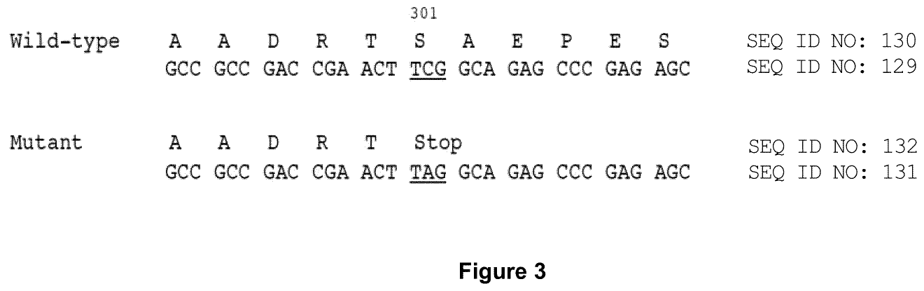

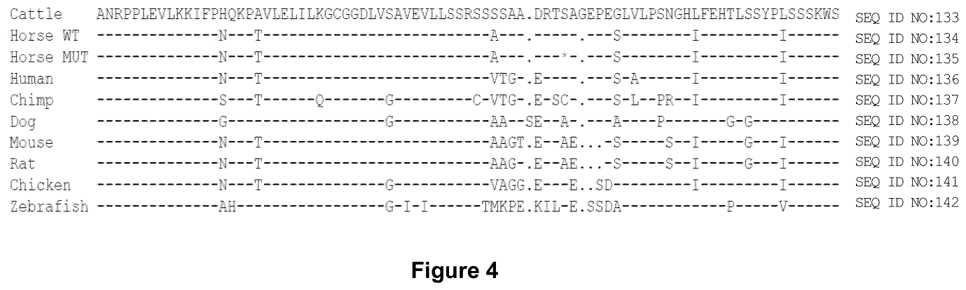

A nonsense mutation located in exon 2 of DMRT3 shows complete concordance with the ability to pace. The critical interval for the Gait locus comprises the four genes DMRT1, DMRT2, DMRT3 and GTF2A2. The DMRT genes belong to a family of transcription factors that contains the zinc-finger like DNA binding DM domain (Murphy et al. 2007. Vertebrate DM domain proteins bind similar DNA sequences and can heterodimerize on DNA. BMC Mol. Biol. 8:58). We sequenced most of the DMRT exons in this region and identified a small number of sequence polymorphisms (Table 4). One of these (DMRT3.3), located in exon 2 of DMRT3 at nucleotide position 22,999,655, caused a nonsense mutation in the allele associated with the ability to pace (FIG. 3). Thus, the gait allele is predicted to encode a truncated form of the DMRT3 protein (SEQ ID NO: 4) lacking the last 174 amino acids, reducing the total size of the protein from 474 to 300 amino acids. Full length wild-type horse DMRT3 is shown as SEQ ID NO: 2. An alignment of the part of the DMRT3 protein including the mutated amino acid position 301 (Serine) in horses shows that this protein is highly conserved among vertebrates including fish, bird and mammalian species (FIG. 4).

TaqMan assays were designed for the polymorphisms at nucleotide positions 22,967,656 (the most significantly associated SNP in the GWA analysis) and at position 22,999,655 (the mutation in DMRT3 creating a premature Stop codon). These were used to screen all 70 Icelandic horses included in this study. Both SNPs showed complete association between homozygosity for the non-reference allele at both loci and the phenotype (Table 5), the statistical support for an association was overwhelming (P=6.73×10⁻¹⁰ for both SNPs, Fisher's Exact Test). The results imply that there is very strong linkage disequilibrium between these two SNPs in the studied population, the two SNPs are located 32 kilo base pairs apart. Nine animals that were classified as four-gaited were homozygous for the haplotype associated with the gait allele (Table 5). These animals were either misclassified by their owners, which is fully possible, or the Gait genotype shows incomplete penetrance due to interaction with environmental factors (for instance training) or other unknown genetic factors.

We tested 2 North-Swedish Trotters and 61 Swedish Standardbred horses (both used for harness racing in Sweden) to investigate if the gait allele is present in other horse breeds. We found that both the 2 North-Swedish Trotters and 59 Standardbred horses were homozygous for the DMRT3 nonsense mutation at nucleotide position 22,999,655 on horse chromosome 23 while the remaining 2 Standardbred horses were heterozygous A/C. The high frequency of this allele in these breeds strongly suggests that the mutation has a favourable effect on the ability to trot at a fast speed. In deed, the two horses identified as being heterozygous for the gait allele were also considered as being poor trotters. We predict that the gait allele is present at a high frequency in most, if not all, gaited horse breeds as well as horses used for harness racing.

TABLE 5
Highly significant association between SNPs at nucleotide
position 22,967,656 (BIEC2-620109) and 22,999,655 (DMRT3.3)
on horse chromosome 23 in relation to the phenotypic classification
of Icelandic horses as four-gaited or five-gaited. Statistics
was calculated using Fisher exact test, with the Gait
allele as the recessive allele.

| | | |
|---|---|---|
| MARKER | BIEC2-620109 | DMRT3.3 |

| | | | |
|---|---|---|---|
| Allele 1 (A₁) | | C | C |
| Allele 2 (A₂) | | T | A |
| Wild-type | A₁/— | 21 | 21 |
| | A₂/A₂ | 9 | 9 |
| | A₁/— | 1 | 1 |
| Five-gaited | A₂/A₂ | 39 | 39 |
| p | | 6.73E−10 | 6.73E−10 |
| OR | | 83.18 | 83.18 |

A₁/— = A₁/A₁ or A₁/A₂
OR = odds ratio.

TABLE 6
Genotype distribution for a nonsense mutation
(A) in DMRT3 among horse populations.

| Breed | Number | CC | CA | AA |
|---|---|---|---|---|
| Icelandic Horse | 70 | 0.01 | 0.30 | 0.69 |
| Standardbred Trotter | 61 | 0.00 | 0.03 | 0.97 |
| Cold Blooded Trotter | 2 | 0.00 | 0.00 | 1.00 |

TABLE 7
SNP sequences

| SEQ | | | |
|---|---|---|---|
| ID | | | |
| NO | sequence | SNP | position |
| SEQ | TTGTTGGGGTCTTATGCAAA | BIEC2_ | 22 967 656 |
| ID | GTGCAGAAATAGTCTTTTGG | 620109 | |
| NO: | AAAAACGTAC[C/T]GTTTG | | |
| 5 | CACTATTTTCTTATTTCTAT | | |
| | TCACCCTTAATGTGAACCAT | | |
| | TCCAA | | |
| SEQ | AGAAATGATATATAAAAATT | BIEC2- | 22 628 976 |
| ID | ACGAATGCCTCTTAGACAGA | 619907 | |
| NO: | ATCCTTATGT[A/G]TGGCA | | |
| 6 | CAGAAGTATTTAGTTCGCTT | | |
| | AACAGATATTGAGTGCTTAT | | |
| | ATGAG | | |
| SEQ | CTCTTCCTTGCATCCTATCC | BIEC2- | 23 315 071 |
| ID | CCCTAGTGTCGCAAGGGAAG | 620244 | |
| NO: | TTGTGAGAGA[C/T]GAGCT | | |
| 7 | TGTAGATCTGCTCTAGAAAA | | |
| | TAGGCCTGTTTTCTTAAGAA | | |
| | ACCGT | | |
| SEQ | CAGAGTGCCGGTCTGTGGCT | ANKRD15.1 | 22 793 939 |
| ID | GTGGGCGCTGACGAGCACAT | | |
| NO: | GGACAACATT[G/C]TCGTG | | |
| 8 | TACCACAGGGGCTCCAGGTC | | |
| | CTGTAAGGATGCTGCTGTGG | | |
| | GGACA | | |
| SEQ | AGAACTCATTCAAAACCACC | ANKRD15.2 | 22 810 322 |
| ID | AGGCTTACTAGGCTTTTTTA | | |
| NO: | AATAGACTTG[A/G]CTTTG | | |
| 9 | AACTTCTAAGTGCAGGATCT | | |
| | AAAACCACTGGCGAAATTTC | | |
| | TGGAA | | |
| SEQ | TTACCTGCATGCCTCTCCCC | ANKRD15.3 | 22 812 345 |
| ID | CTAAACCATTTCTAGCATGT | | |
| NO: | GTGGGCAGAG[T/G]GGGCA | | |
| 10 | TCGTGCTGCCCTGCTCACTG | | |
| | GATCACTCTGGGAACGTTTC | | |
| | CTTCA | | |
| SEQ | AAGGATATGGTGAGTCTGAC | ANKRD15.4 | 22 812 251 |
| ID | CTACAGACACTGTCCCCGGT | | |
| NO: | CTGTACAAAG[T/C]GCCCA | | |
| 11 | AGTGGTGACAAAGCATCCCT | | |
| | CGCCTGCCCCCTGAGCTGTT | | |
| | ACCTG | | |
| SEQ | AACGCCAAAGCCAGCCAGGT | ANKRD15.5 | 22 818 132 |
| ID | GACTGCGCTTGCTTCCTGGG | | |
| NO: | CTCATGCTCA[C/T]ACTGC | | |
| 12 | TGTGACCCGCACAGGTGCCC | | |
| | ACGCCACACTTCCCACCGCT | | |
| | CGGCA | | |
| SEQ | GCTTGCTTCCTGGGCTCATG | ANKRD15.6 | 22 818 158 |
| ID | CTCACACTGCTGTGACCCGC | | |
| NO: | ACAGGTGCCC[A/G]CGCCA | | |
| 13 | CACTTCCCACCGCTCGGCAC | | |
| | TCACTCATGGCCCAGCCCCG | | |
| | AGTCC | | |
| SEQ | ACTGAATGTATACATTTTGT | ANKRD15.7 | 22 821 872 |
| ID | GCCTGAACTCACCAGCAAAC | | |
| NO: | AGAAGGCAGA[A/C]AACCA | | |
| 14 | AGGGTTGAAGGCTGGAGCTG | | |
| | TCACAGTAGAAGTTGAGCCA | | |
| | GCAGG | | |
| SEQ | CATTTTGTGCCTGAACTCAC | ANKRD15.8 | 22 821 884 |
| ID | CAGCAAACAGAAGGCAGAAA | | |
| NO: | ACCAAGGGTT[G/C]AAGGC | | |
| 15 | TGGAGCTGTCACAGTAGAAG | | |
| | TTGAGCCAGCAGGAATTTGC | | |
| | TGGCC | | |
| SEQ | TCACTCTAATCAAGTTGCTA | SNP.1 | 22 868 190 |
| ID | TCACCATTCACACAATTGTC | | |
| NO: | CAGGATAGTA[C/T]TGGGA | | |
| 16 | CCCCAGAAAGATCACGCCGC | | |
| | TCCATTCCCATTTCCCACTT | | |
| | GTTCC | | |
| SEQ | CTGGGCTGAAACAGGTGGTC | SNP.2 | 22 868 678 |
| ID | CTGCTTTCCCCGCCTGCCTG | | |
| NO: | GTCAGGCTGC[G/A]CTCTT | | |
| 17 | CTCCCCTCCCCAGGCTTAAG | | |
| | TCACTTCATGCAGAACCCTT | | |
| | TATAC | | |
| SEQ | CCAGCATTCTCCGCTTTCAA | SNP.3 | 22 872 820 |
| ID | CTTTCTCCCGCTCCTCCAAT | | |
| NO: | CCAAACTGGA[G/C]TTAGC | | |
| 18 | ATCAGCTACCCACAATGATC | | |
| | AAGCATTTTCTGTGTGGCAG | | |
| | GCCTG | | |
| SEQ | AGGCAAGAAGCGATAGGCAA | SNP.4 | 22 876 848 |
| ID | AAGGCAGCAAGAGCTGGACC | | |
| NO: | TGCAGATTTG[C/A]AAGTT | | |
| 19 | CTCTGGAGCCAGTAGGTGGA | | |
| | AACCTCATCAGCAAATGAAC | | |
| | GCAGG | | |
| SEQ | CCACACTGAGAGTCTTATTT | SNP.5 | 22 877 015 |
| ID | GCTGATAGAAATGCAGAGAC | | |
| NO: | TTCTCTTTTC[T/C]GAGGC | | |
| 20 | TTTCAACCTCGTACTTAATT | | |
| | CTCCTAAGTGAGAAAGAAAC | | |
| | CACTC | | |
| SEQ | ACCAGCGGGAGACTGAGGCT | DMRT3.1 | 22 986 593 |
| ID | GCGAGCGCCGCAAAGACGGG | | |
| NO: | TGCCGCATCT[C/T]TGGCC | | |
| 21 | AGCCCGGAGCGCACGCGGCC | | |
| | GCCGGAGCTGCGGGACCAAG | | |
| | GACCG | | |
| SEQ | CCGTCTCAGCCGCCGCCGCC | DMRT3.2 | 22 987 143 |
| ID | GCAGCGTCCCGCCGCCGAGT | | |
| NO: | TGGCTGCGGC[C/T]GCCGC | | |
| 22 | GCTGCGCTGGGCCACCGAGC | | |
| | CGCAGCCCGGGGCGCTGCAG | | |
| | GCGCA | | |
| SEQ | GGAGGTCCTCCTCTCCAGCC | DMRT3.3 | 22 999 655 |
| ID | GCTCCTCGGCCTCGGCCGCC | | |
| NO: | GACCGAACTT[C/A]GGCAG | | |
| 23 | AGCCCGAGAGCCTCGTGTTG | | |
| | CCCTCCAACGGGCACATCTT | | |
| | TGAAC | | |
| SEQ | CTCTCCAGCCGCTCCTCGGC | DMRT3.4 | 22 999 665 |
| ID | CTCGGCCGCCGACCGAACTT | | |
| NO: | CGGCAGAGCC[C/G]GAGAG | | |
| 24 | CCTCGTGTTGCCCTCCAACG | | |
| | GGCACATCTTTGAACACACC | | |
| | TTGAG | | |
| SEQ | GGCCTGGCCCCTAGGGCATT | SNP.6 | 23 009 648 |
| ID | GAAGGGCTGGGGAGAGTCAC | | |
| NO: | ATGTACTCCC[A/T]CTGTG | | |
| 25 | GCCTGAAGACCTACCTGGAG | | |
| | GGAAACCAGCTTGCTTAGGG | | |
| | GGCCT | | |

TABLE 8
Sequence variants on horse chromosome 23 showing strong genetic
association with the Gait mutation in horses. The Gait mutation
occurs on horse chromosome 23, nucleotide position 22,999.655
bp and is indicated in bold italics below.

| | Location/ | Coordinate | Ref. | Var. |
|---|---|---|---|---|
| Type | consequence ¹ | (EquCab2) | Allele² | allele(s)³ |
| SNP | intronic | 22919878 | A | G |
| SNP | intronic | 22920361 | C | T |
| SNP | intronic | 22920434 | A | T |
| SNP | intronic | 22920646 | G | A |
| SNP | intronic | 22920717 | C | T |
| SNP | intronic | 22921203 | G | T |
| SNP | intronic | 22922079 | A | G |
| SNP | intronic | 22922780 | C | T |
| SNP | intronic | 22923569 | A | G |
| SNP | intronic | 22924120 | G | A |
| INDEL | intronic | 22924142 | — | A |
| SNP | intronic | 22924299 | T | G |
| SNP | intronic | 22924380 | A | G |
| SNP | intronic | 22924407 | C | T |
| SNP | intronic | 22926098 | C | T |
| SNP | intronic | 22926188 | T | C |
| SNP | intronic | 22926872 | A | C |
| SNP | intronic | 22927387 | C | T |
| SNP | intronic | 22927607 | T | C |
| SNP | intronic | 22928220 | C | T |
| SNP | intronic | 22928537 | T | G |
| SNP | intronic | 22928587 | A | G |
| SNP | intronic | 22929137 | G | A |
| SNP | intronic | 22930011 | A | C |
| SNP | intronic | 22932024 | G | A |
| SNP | intronic | 22932895 | A | G |
| SNP | intronic | 22933218 | A | G⁴ |
| SNP | intronic | 22936034 | A | G |
| SNP | intronic | 22940759 | T | G |
| SNP | intronic | 22942423 | T | A |
| SNP | intronic | 22945643 | G | C |
| SNP | intronic | 22946599 | A | T |
| SNP | intronic | 22948774 | C | T |
| SNP | intronic | 22949055 | A | G |
| SNP | intronic | 22949108 | A | G |
| SNP | intronic | 22949240 | T | C |
| SNP | intronic | 22949710 | A | G |
| SNP | intronic | 22956846 | G | T |
| SNP | intronic | 22960132 | A | C |
| SNP | intronic | 22960528 | T | C |
| SNP | intronic | 22960710 | C | T |
| SNP | intronic | 22964042 | C | T |
| INDEL | intronic | 22965059 | — | GA |
| SNP | intronic | 22967119 | C | T |
| SNP | intronic | 22967656 | C | T |
| SNP | intronic | 22967915 | G | C |
| SNP | intronic | 22968898 | G | A |
| SNP | intronic | 22973984 | C | T |
| SNP | intronic | 22974589 | T | C |
| SNP | intergenic | 22979124 | T | C |
| SNP | intergenic | 22980014 | C | T |
| SNP | intergenic | 22982879 | T | C |
| INDELs | intergenic | 22984588 | A | — |
| INDEL | intergenic | 22985746 | G | — |
| SNP | intronic | 22988210 | C | A |
| SNP | intronic | 22988991 | T | G |
| SNP | intronic | 22993092 | C | A |
| SNP | intronic | 22994591 | C | A |
| SNP | intronic | 22999058 | G | A |
| SNP | stopgain SNP | 22999655 | C | A |
| SNP | intergenic | 23002606 | A | G |
| INDEL | intergenic | 23003956 | — | TG |
| SNP | intergenic | 23008772 | G | A |
| SNP | intergenic | 23008789 | G | A |
| SNP | intergenic | 23009648 | A | T |
| SNP | intergenic | 23010164 | G | A |
| SNP | intergenic | 23011289 | G | C |

¹ Location: Indicates where the SNP is located in relation to Ensembl genes. In cases of coding sequence overlap, the predicted consequence to the protein is indicated. The gene intersection was performed using the software ANNOVAR.
²Ref. allele. This is the reference allele in the horse genome assembly (EquCab2).
³Var. allele: This is the variant allele at polymorphic position showing very strong association with the Gait mutation. For insertion polymorphisms in relation to the reference assembly (EquCab2), the reference allele is denoted “—” and for deletions in relation to the reference the variant allele is denoted “—”. The sequenced mutant horse was homozygous for the variant allele at all sites except one (see Footnote 4) listed in this table unless otherwise stated in the Var. allele column.
⁴This SNP was identified as heterozygous (AG) in the mutant horse and homozygous for the reference allele in the control horse. The G-allele at this SNP has likely occurred subsequent to the DMRT3 nonsense mutation.

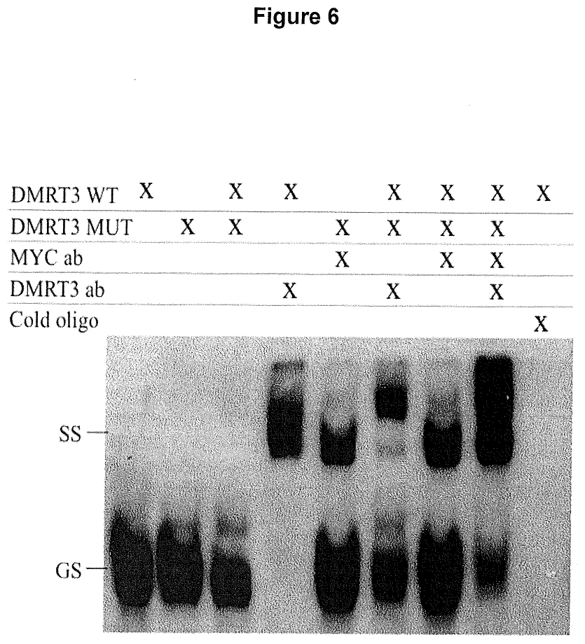

Electrophoretic mobility shift assays (EMSA). The oligonucleotide 5′-ggatccTCGAGAACAATGTAACAATTTCGCCC-3′ (SEQ ID NO: 34) and its complementary sequence were annealed in 10 mM Tris pH 7.5, 1 mM EDTA, 50 mM KCl by firstly heating to 95° C. for 2 min and thereafter cooled to 25° C. (2 min/degree). The duplex was labelled with Klenow DNA polymerase and [α-32P]-dCTP and purified using a Bio-Rad Micro Bio-Spin 30 column. DMRT3 wild type and mutant protein were produced by in vitro-translation using a TNT Quick Coupled Transcription/Translation System (Promega). EMSA was performed as described by Culbertson & Leeds, 2003 (Looking at mRNA decay pathways through the window of molecular evolution. Curr. Opin. Genet. Dev. 13, 207-214) with the following modifications. No plasmid DNA was added and 1.0 μl in vitro-translated protein and 150× cold competitor were used. The reaction mixture was incubated on ice for 20 min before adding the radioactive oligo and thereafter incubated at room temperature for 30 min. Gels were run at 150 V in room temperature. Both full-length wild-type and mutant DMRT3 protein were found to bind a previously defined DMRT-binding motif (FIG. 6). Thus, the DMRT3 mutation does not lead to an altered expression pattern and the mutant protein appears to maintain its cellular localization and DNA binding profile. It may therefore be a dominant negative form with normal DNA-binding but defective interaction with other proteins. This would be consistent with the clear phenotypic effects observed in heterozygotes. However, the mutation is not fully dominant as CA heterozygotes and AA homozygotes show distinct phenotypic differences.

### Conclusions

We have presented abundant evidence that the DMRT3_Ser301STOP mutation has a major effect on gaits in horses. Our interpretation of the phenotypic consequences of this mutation is that homozygosity for the mutation is required but not sufficient for pacing, as many Standardbred Trotters and some Icelandic horses that are homozygous mutant do not pace. On the other hand heterozygosity or homozygosity for the mutation are permissive to enable a variety of four-beat ambling gaits to be performed, with genetic modifiers that may be unique to each gaited breed. The mutation promotes ambling gaits and pace and it inhibits the transition from trot or pace to gallop, which explains its high frequency in pacers and trotters used for harness racing. It is an open question if the mutation alters the fate of DMRT3-neurons or changes their transcriptional regulation, but it is clear that these neurons must have a key role for the control centre in the spinal cord coordinating limb movements.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for predicting the pattern of locomotion in a horse including the ability to use alternative gaits, to trot or pace at a fast speed, and to perform in dressage, said method comprising steps of;
i) extracting DNA from a sample obtained from a horse, and
ii) determining in said DNA the presence or absence of the nonsense mutation in exon 2 of the DMRT3 gene at nucleotide position 22,999,655 on horse chromosome 23, said nucleotide position corresponding to nucleotide position 939 in SEQ ID NO:3, and position 51 in SEQ ID NO:23, wherein said determining comprises contacting a nucleotide primer that specifically binds to the DNA sequence between nucleotide positions 22,628,976 and 23,315,071 base pairs on horse chromosome 23 or to the complementary strand thereof with said extracted DNA under hybridizing conditions and detecting the presence of a hybridization product comprising the primer and said DNA,
wherein the nucleotide positions on horse chromosome 23 refer to the horse reference sequence according to the September 2007 Equus caballus draft assembly EquCab2.

2. The method according to claim 1, wherein said nucleotide primer specifically binds to the DNA sequence between nucleotide positions 22,919,878 and 23,011,289 base pairs on horse chromosome 23.

3. The method according to claim 1, wherein said nucleotide primer specifically binds to the sequences SEQ ID NO:1, 3 and 5-25.

4. The method according to claim 1, wherein said nucleotide primer is selected from SEQ NO:26, SEQ ID NO:27, SEQ ID NO:30, and SEQ ID NO:31.

5. The use of the method according to claim 1 for selection a horse for breeding.

6. The use of the method according to claim 1 for paternity testing.

7. The method according to claim 1, wherein said nucleotide primer specifically binds to the DMRT3 gene.

8. The method according to claim 1, wherein said determining is performed by polymerase chain reaction (PCR), allele specific hybridization, a 3′exonuclease assay, a Taqman assay, fluorescent dye and quenching agent-based PCR assay, allele-specific restriction enzymes (RFLP-based techniques), direct sequencing, the oligonucleotide ligation assay (OLA), pyrosequencing, the invader assay, minisequencing, DHPLC-based techniques, single strand conformational polymorphism (SSCP), allele-specific PCR, denaturating gradient gel electrophoresis (DGGE), temperature gradient gel electrophoresis (TGGE), chemical mismatch cleavage (CMG), heteroduplex analysis based system, techniques based on mass spectroscopy, invasive cleavage assay, polymorphism ratio sequencing (PRS), microarrays, a rolling circle extension assay, HPLC-based techniques, extension based assays, ARMS (Amplification Refractory Mutation System), ALEX (Amplification Refractory Mutation Linear Extension), SBCE (Single base chain extension), molecular beacon assays, invader assays, ligase chain reaction assays, 5′-nuclease assay-based techniques, hybridization capillary array electrophoresis (GAE), and solid phase hybridization, dot blots, reverse dot blots, and chips.

9. The method according to claim 1, wherein said nucleotide primer specifically binds to the sequence SEQ ID NO: 18.

10. The method according to claim 1, further comprising selecting the horse for breeding based on a detection of the presence of the hybridization product.
